(19) United States
(12) Patent Application Publication
(10) Pub. No.: US 2005/0136487 A1
(43) Pub. Date: Jun. 23, 2005

(54) Transmissible spongiform encephalopathy detection in cervids, sheep and goats

(76) Inventors: Donald W. Meyer, Ixonia, WI (US); Katherine I. O'Rourke, Pullman, WA (US)

Correspondence Address: REINHART BOERNER VAN DEUREN S.C.;ATTN: LINDA GABRIEL, DOCKET COORDINATOR, 1000 NORTH WATER STREET, SUITE 2100, MILWAUKEE, WI, 53202, US

(21) Appl. No.: 10/974,471

(22) Filed: Oct. 27, 2004

Related U.S. Application Data
(60) Provisional application No. 60/515,803, filed on Oct. 27, 2003.

(51) Int. Cl. G01N 33/53; G01N 33/567; G06F 19/00; G01N 33/48

(52) U.S. Cl. 435/7.2; 702/19; 435/40.5

(57) ABSTRACT

A method for diagnosing scrapie in sheep or goats and chronic wasting disease in deer or elk is provided including detecting spectral changes between disease-positive samples and disease-negative samples providing a calibration model for use in classifying unknown samples as disease-positive or disease-negative.

### CROSS REFERENCE TO RELATED APPLICATIONS

This application claims priority of provisional application Ser. No. 60/515,803, which is entitled “Method of Detecting Transmissible Spongiform Encephalopathies”, and which was filed on Oct. 27, 2003, the entirety of which is hereby incorporated herein by reference.

### BACKGROUND OF THE INVENTION

Field of the Invention

The present invention relates to methods for detecting a change in spectral response between normal and diseased tissue and/or fluid samples as an indication of chronic wasting disease and/or scrapie, and more particularly, to a method of identifying a transmissible spongiform encephalopathy infection in a deer, elk, sheep or goat subject by comparing spectral data obtained from a sample taken from the cervid, sheep or goat subject to a calibration model including spectral data obtained from known TSE-normal and TSE-diseased samples obtained from the same species as the subject.

Transmissible spongiform encephalopathies (TSEs), also known as prion diseases, are a family of contagious, degenerative neurological diseases found in humans and animals. A TSE infects the central nervous system of a host species and eventually attacks the brain of the host species. TSEs have a long incubation period, and once infected, a host species may not exhibit clinical symptoms for years. Clinical symptoms of TSEs can include emaciation, loss of bodily condition, loss of coordination and movement, excessive salivation, and/or loss of bodily functions. While the clinical symptoms of each specific transmissible spongiform encephalopathy (TSE) may vary somewhat from species to species, TSEs always cause death in infected victims.

TSEs are characterized by tiny holes or lesions in the brain that give the brain a sponge-like appearance and are associated with the aggregation in the brain of prions, an abnormal form of a naturally occurring, membrane protein called a prion protein (PrP). Normal prion protein molecules, designated as PrP or PrP^{c} (cellular), are predominantly found on the surface of neuronal cells and in spinal fluid as well as on the surface of certain other non-neuronal cell types such as lymphoreticular tissue. The normal PrP^{c} is sensitive to digestion with specific enzymes.

Disease-associated prions are defined as small, proteinaceous infectious particles that resist inactivation by procedures that modify nucleic acids. These infectious prions, designated PrP^{res} (protease resistant protein), are characterized as abnormal, insoluble isoforms of normal mammalian PrP^{c}.

Normal PrP^{c} and infectious PrPᵣₑₛ share the same amino acid sequence and have almost identical molecular weights of 33 to 35 kd (6033858), but differ from each other in their secondary structure. Normal prion proteins contain a predominantly alpha helix structure and contain almost no beta sheet structure. It is believed that the difference between normal PrP^{c} and infectious PrP^{res} is conformational, with infectious PrP^{res} containing a significantly higher beta-sheet content than normal PrP^{c}. The normal prion protein's usual function is unknown, but its shape can be converted into the abnormal, beta-sheet rich disease-associated form. Once the abnormal prions are present, it is suggested that the abnormal prion protein spreads in a chain reaction by converting normal prion molecules into the abnormal form, i.e. converting PrP^{c} to PrP^{res}.

The pre-clinical phase of a TSE is the long incubation period after infection with PrP^{res} in which the species exhibits no external clinical symptoms of the disease. However, it is known that the presence of PrP^{res}, or a precursor thereof, can be detected in the lymphoreticular system and in lymphoeticular tissues such as the tonsils, lymph nodes, eyes, spleen, or blood in mammalian species susceptible to TSEs. For example, because infectious prions, PrP^{res}, are protease resistant, it is suggested that prions accumulate in the lymph system or nervous system of an animal species, in addition to the brains of infected the host, where they aggregate into distinctive filaments or plaques on the surface of the cell membrane.

Prions are transmitted from one host to another; however, they do not exhibit many other features of traditional infective agents, such as bacteria, viruses, or fungi. As such, prions are referred to as infectious “agents” of disease. How a prion moves from species to species is somewhat unclear; however, it is known that a TSE can be transmitted from animal to animal contact, and that animals exposed to a TSE contaminated environment, infected tissue, body fluids, infected carcasses, or contaminated medical instruments may also become infected. Once the infectious agent enters the brain of the host, it can lie dormant for several years, but when it is activated, the abnormal prion molecules kill brain cells leaving large areas of spongy holes in the brain, and leaving large clumps of abnormal prion protein in the brain. Once the abnormal prion is activated, the disease can run its course in less than a year, causing death. Further, it has also been discovered that routine sterilization procedures, such as boiling or irradiation, do not prevent transmission of TSEs. Further, cleaning chemicals that one would use to disinfect surfaces for bacteria or viruses such as Lysol, or Betadine, are not effective in destroying the infectious capability of the prions.

Scrapie, a disease that has been well known for several centuries, is the infective TSE found in goats or sheep. Scrapie is thought to be spread from the ewe to her offspring and to other lambs through contact with the placenta and placental fluids. In addition, it is believed that some sheep carry a genetic predisposition for susceptibility to a scrapie infection. Scrapie infected flocks of sheep or goats that contain a high percentage of susceptible animals can experience significant losses. As the number of infected animals increases, the age at onset of clinical signs decreases, making these flocks economically unviable. In addition, the presence of scrapie in the United States prevents the export of breeding stock, semen, and embryos to many other countries.

Chronic wasting disease (CWD) is a TSE found in free ranging and captive mule deer, white tailed deer and elk across North America. CWD is of particular concern because the presence of CWD across the United States and Canada is growing. In addition, CWD is of particular concern in the United States where deer hunting is a traditional part of recreational sporting and tourism in several states where infected deer and elk have been discovered. In addition, CWD has affected wildlife industries as well as domestic and international markets for farmed cervids and cervid products.

Moreover, it has been suggested that food products containing TSE infected tissue may cause cross-species forms of a transmissible spongiform encephalopathy disease. For example, it is believed that in the 1980's, feed contaminated with scrapie-infected sheep tissue and/or sheep by-products was fed to cattle causing infection. It is unknown whether humans can be infected with a TSE by ingesting CWD infected meat from deer or elk. However, the occurrence of new variations of TSEs and the spread of CWD in the United States has emphasized the need for a reliable and efficient test for the disease.

Current Methods of Detection

Current diagnosis of Chronic wasting disease or scrapie involves several different methodologies.

Postmortem microscopic or histological examination of brain tissue, especially the medulla oblongata at the obex, is still routinely performed as a method of confirming a CWD or scrapie case. However, such method requires the expertise of laboratory technicians and is time consuming, and therefore, not a practical method of testing a large volume of tissue samples. In addition, microscopic examination of brain tissue detects lesions in the brain occurring only after the onset of the clinical disease, but is not used to detect early stages of infection with the disease.

The Western Blot assay test for the detection of CWD or scrapie relies on the differential sensitivity of the infectious prions, PrP^{res}, to protease K and thus, can be a quantitative measure of the presence of infectious prions. Tissue samples, for example, brain or lymph tissue, are first digested with protease K or an antibody that can discriminate between normal cellular PrP^{c} of the specific host species and the protease resistant form PrP^{res}. The sample is denatured and diluted to an end point where PrP^{res} can no longer be detected in SDS PAGE gel. While Western Blot is considered a reliable, gold standard test for the detection of prion proteins, and therefore, a diagnosis for CWD, the Western Blot test is considered technically demanding, involves careful and inefficient preparation of samples, and required the use of expensive and environmentally sensitive monoclonal antibodies. Accordingly, improper analytical techniques, instrumentation malfunctions, biased calculations, or inconsistent sample preparation techniques from technician to technician can result in errors in the test, and therefore, the possibility in error in test results. Because of these difficulties and because Western Blot testing cannot be automated, it is not an appropriate test for the routine diagnosis, confirmation or screening of samples for a TSE in large populations of deer, elk, sheep or goat.

CWD testing is also performed by immunohistochemistry assay of formalin fixed tissues. Although IHC is a sensitive and specific diagnostic test, the assay requires careful collection and trimming of tissues, formalin fixation, processing and sectioning, IHC assay, and interpretation of results by a pathologist. The turnaround time is minimally measured in days and laboratory capacity is limited. In addition, tissue preparation is lengthy and subject to operator error. As with the Western Blot assay, use of expensive PrP^{res} specific monoclonal antibodies is a further disadvantage to implementing this type of testing in a large-scale application. Further, the nature of IHC testing makes diagnosis of CWD at the early stages of the disease subject to operator error or inconsistency from operator to operator.

Newer methods include, for example, the tonsillar biopsy method, in which tonsil tissue is collected from live or deceased deer or elk and tested using either of the aforementioned assays. This method is advantageous because it can be performed on live or quarantined deer, and because it can be used to detect the presence of CWD at an earlier stage of infectivity. However, the method involves racking, capturing, sedating and sampling individual deer and/or elk in infected areas or zones, making widespread application of the method expensive and time consuming to execute.

With respect to scrapie, commercial testing to determine the genetic makeup of sheep, with respect to the mutation of the prion gene, at codons 136 and 171, is available in the United States. Indeed, genetic testing for susceptibility is currently being practiced by many breeders. However, such genetic testing can be very costly and time consuming.

IR spectroscopy can be used to measure the absorbance/reflectance of infrared light in a sample. Specific absorbance peaks can be correlated to the presence of specific functional groups, chemical bonds and/or structural features of a compound in the sample, including water, proteins, or lipids. The absorbance patterns of samples can be compared to a reference (calibration) set to identify peaks specifically associated with a group, bond or structural feature of interest. IR analysis, alone or in conjunction with a Fourier transform function (FTIR), is a standard tool in some prion research laboratories, in which the difference in protein conformation between the normal cellular prion protein and the disease-associated prion were detected. Diagnostic use of the method has been investigated in a hamster model of scrapie. However, application of the technique to naturally occurring chronic wasting disease or scrapie has not been described. Moreover, these applications utilize FTIR microscopy in conjunction with an FTIR spectroscopy instrument, requiring time consuming sample preparation, long scanning times and significant operator training. Further, in conjunction with the prior, the cost of FTIR microscopy equipment and required accessories discourages routine, widespread use of these methods in the diagnosis of CWD or scrapie.

### BRIEF DESCRIPTION OF THE DRAWINGS

FIG. 1A is a schematic structure of a lymph node, identifying the cortex and paracortex regions and the primary and secondary follicles contained therein and illustrating a preferred method of sectioning a lymph node to expose the cortex or paracortex without exposing medulla sections;

FIG. 1B is a schematic structure of a sheep brain and brain stem, identifying the obex portions utilized in the present invention;

FIG. 2 illustrates the absorbance spectra (4000 cm⁻¹ to 500 cm⁻¹) from for a lymph tissue sample taken from a known positive (CWD-infected) white tailed deer subject;

FIG. 3 illustrates the absorbance spectra (4000 cm⁻¹ to 500 cm⁻¹) from for a lymph tissue sample taken from a known negative (disease free) white tailed deer subject;

FIG. 4 is a scatter plot of the Mahalanobis distances for each calibration sample within the calibration set of Example 1, illustrating two distinct clusters of tissue samples, corresponding to a “positive” cluster of tissue samples and a “negative” cluster of tissue samples;

FIGS. 5A and 5B are calibration results tables of the cross-validated calibration data of Example 1 and plotted in FIG. 4;

FIG. 6 is a scatter plot of the Mahalanobis distances for each validation sample, in addition to the calibration samples, in Example 1 demonstrating alignment with either the “positive” cluster or “negative” cluster of the calibration data set;

FIGS. 7A and 7B are validation results tables of the validation set data of Example 1 and plotted in FIG. 6;

FIG. 8 is a scatter plot of the Mahalanobis distances for each calibration sample within the calibration set of Example 2, illustrating two distinct clusters of tissue samples, corresponding to a “positive” cluster of tissue samples and a “negative” cluster of tissue samples;

FIG. 9 is a scatter plot of the Mahalanobis distances for each validation sample, in addition to the calibration samples, in Example 2 demonstrating alignment with either the “positive” cluster or “negative” cluster of the calibration data set of Example 2;

FIG. 10 is a scatter plot of the Mahalanobis distances for each calibration sample within the calibration set of Example 3, illustrating two distinct clusters of tissue samples, corresponding to a “scrapie positive” cluster of tissue samples and a “scrapie negative” cluster of tissue samples;

FIG. 11 is a calibration results table of the cross-validated calibration data of Example 3 and plotted in FIG. 10;

FIG. 12 is a scatter plot of the Mahalanobis distances for each validation sample in Example 3 demonstrating alignment with either the “scrapie positive” cluster or “scrapie negative” cluster of the calibration data set;

FIG. 13 is a validation results table of the validation set data of Example 3 and plotted in FIG. 12;

FIG. 14 is a scatter plot of the Mahalanobis distances for each calibration and validation sample in Example 4 demonstrating a “scrapie positive” cluster or “scrapie negative” cluster for whole blood samples;

FIG. 15 is a results table presenting the calibration and validation results of Example 4 and plotted in FIG. 14;

FIG. 16 is a scatter plot of the Mahalanobis distances for each calibration and validation sample in Example 5 demonstrating a “scrapie positive” cluster or “scrapie negative” cluster for blood serum samples;

FIG. 17 is a results table presenting the calibration and validation results of Example 5 and plotted in FIG. 16;

FIG. 18 is a results table presenting the calculated score for each of the samples in Example 5A;

FIG. 19 is a scatter plot of the Mahalanobis distances for each calibration and validation sample in Example 6 demonstrating a “scrapie positive” cluster or “scrapie negative” cluster for blood plasma samples; and

FIG. 20 is a results table presenting the calibration and validation results of Example 6 and plotted in FIG. 19.

### SUMMARY OF THE INVENTION

In light of the forgoing, there is a need for an efficient, inexpensive and reliable method for detecting chronic wasting disease in cervids (deer and/or elk) or scrapie in sheep or goats, in a high throughput environment in order to increase the efficiency of surveillance and containment efforts. As described herein, any reference made to a transmissible spongiform encephalopathy (TSE) refers to chronic wasting disease (CWD) infections found in deer or elk and scrapie infections found in sheep or goats. In addition, reference to an animal “species” includes deer, elk, sheep and goats and encompasses all genotypical variations/breeds thereof.

Accordingly, it is an object of the present invention to use an IR spectroscopic method, such as Fourier transform infrared spectroscopy (FTIR), as a rapid test for detecting naturally occurring transmissible spongiform encephalopathy infections in live and/or postmortem deer, elk, sheep and/or goats, thereby overcoming various deficiencies and shortcomings of the prior art, including those outlined above. It is a further object of the invention to provide a calibrated spectrometric system for detecting spectral changes between TSE-positive (diseased) and TSE-negative (normal) untreated samples, thereby providing a means for diagnosing CWD in deer or elk and/or scrapie in sheep or goats. It is a related object of the present invention to provide a method for confirming a diagnosis of CWD and/or scrapie infection subsequently obtained using conventional testing methodologies.

Thus, it is a related object of the present invention to provide a method for diagnosing a TSE in any live or postmortem cervid, sheep or goat species that utilizes IR to detect spectral changes between naturally occurring TSE-positive and TSE-negative (fresh or previously frozen) lymph tissue, brain tissue and/or blood substantially untreated samples to provide a calibrated system and method for detecting such TSE in unknown lymph tissue, brain tissue or blood samples of the same species and type.

It is another object of the present invention to provide a primary analytical method for diagnosing a chronic wasting disease and/or scrapie infection that does not destroy the sample, nor requires technically difficult and time consuming mechanical and/or chemical preparation of the sample before a diagnostic result can be obtained. Accordingly, it can be a further object of the present invention to provide a primary test for the diagnosis of CWD or scrapie, wherein a misdiagnosis of such TSE infection is minimized by eliminating systematic and random errors that occur in sample preparation, testing, and interpreting the results of currently known methods. Ergo, it can be an object of the present invention to provide a more reliable and predictable method for diagnosing a CWD or scrapie infection.

It is therefore a further object of the present invention to minimize the costs of analyzing samples for a TSE infection wherein there are no expensive chemicals or antibodies needed in sample preparation, the operators require no special training, and the instrumentation is inexpensive compared to traditional methods of detection. It is a related object of the present invention to reduce the time required to not only prepare the sample, but perform analysis of the sample so that high-volume, rapid throughput testing of samples can be performed easily by governments and researchers. Thus, because there is no subjectivity in the analysis of the spectral information, operator fatigue will not affect the outcome of the analysis.

It will be understood by those skilled in the art that one or more aspects of this invention can meet certain objectives, while one or more other aspects can meet certain other objectives. Each objective may not apply equally, in all instances, to every aspect of the present invention. As such, these objectives can be viewed in the alternative with respect to any one aspect of the present invention.

Other objects, features, benefits and advantages of the present invention will be apparent from the foregoing, in light of the summary and the examples and descriptions which follow, and will be readily apparent to those skilled in the art having knowledge of various prion detection systems, chronic wasting disease and/or scrapie diagnosis methods, conventional sample preparation techniques, and subsequent application thereof. Such objects, features, benefits and advantages will be apparent from the above as taken in conjunction with the accompanying examples, tables, graphs, data and all reasonable inferences to be drawn therefrom.

Without limitation to any particular theory or mode of operation, the spectrometric method described herein identifies one or more constituent groups, functional groups and/or structural features of a molecular compound or composition evidenced by one or more spectra from a diseased sample corresponding to either the presence of the diseased prion protein, PrP^{res}, in the diseased sample, or another chemical, molecular and/or structural alteration occurring within the sample as a precursor to the onset of the clinical disease. Thus, spectral differences between normal and diseased samples can be observed before the TSE induced lesions are observed in the sample. By developing a calibration model for deer, elk, sheep and/or goat animals, these spectral distinctions provide a method for differentially diagnosing a naturally occurring a TSE in unknown samples from that species. The spectrometric method used may be absorption, reflectance, emission or transmission spectrometry. Preferably, absorption or reflectance spectra are obtained from a sample using Fourier transform infrared spectroscopy (FTIR) in order to provide a test wherein there is substantially constant pathlength through the sample, and therefore, more consistency from sample to sample and therefore, in the test results.

Sample Preparation

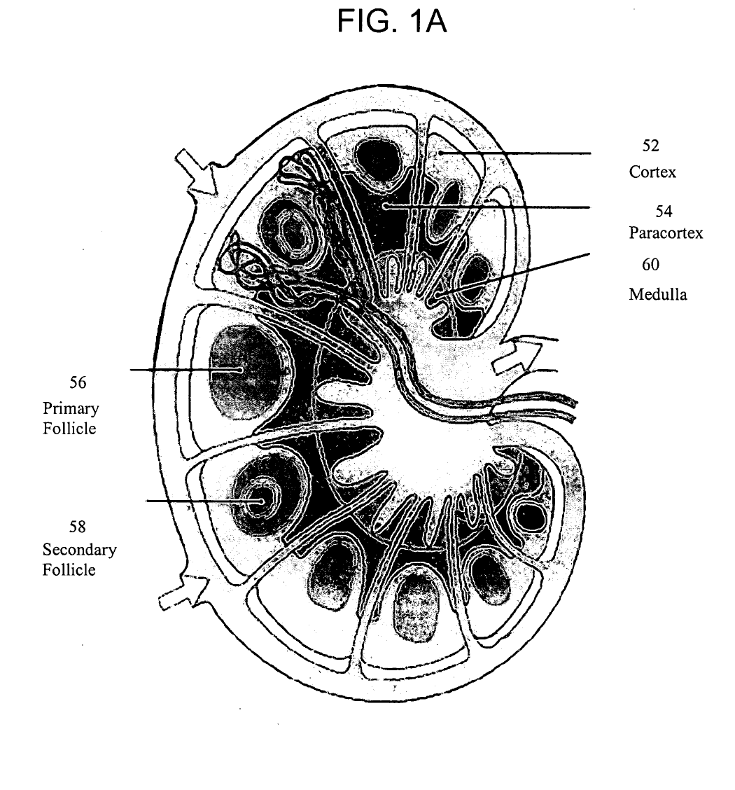

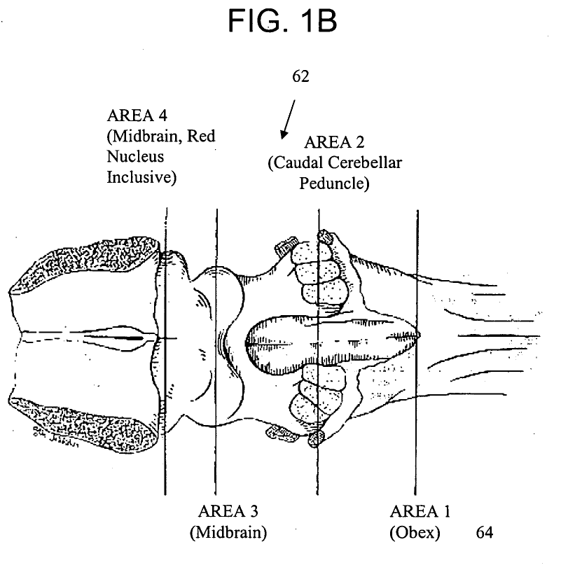

In a first embodiment of the present invention, and with reference to Examples 1 through 3, test samples are obtained from the lymphatic system of a cervid, sheep or goat animal. As illustrated in FIG. 1A, lymph tissue samples are obtained by sectioning a lymph node 50 to expose the cortex 52 or paracortex 54 regions of the node in order to preferably obtain a sample section that includes primarily primary 56 and/or secondary follicles 58, but which minimizes medullary tissue 60 within the sample. Preferably, retropharyngeal node tissue is obtained. It will be apparent to those skilled in the art that any lymphatic system tissue may be used in the present invention, including but not limited to, any lymph node, spleen, lymph nodules (tonsils), thymus, appendix (Peyer's patches) and/or small intestines.

Certain embodiments of the present invention utilize blood samples to practice the methods described herein. (Reference is made to Examples 4 through 6). Blood samples include not only whole blood, but also blood components such as plasma and blood serum. Blood components such as plasma or serum may be separated by conventional methods as are well known to those skilled in the art. Accordingly, the present invention provides a method for detecting chronic wasting disease or scrapie utilizing a blood sample obtained from a live animal subject.

Thus, consistent with the broader aspects of the present invention, other biological fluids known to those skilled in the art to contain PrP^{res} or another TSE-indicating molecular alteration may be utilized. As such, suitable samples can include tears, milk, saliva, semen, or sweat. Further, suitable samples may also include bodily waste such as urine, feces, or menstrual blood.

In other embodiments of the present invention, central nervous system tissues may be utilized. In particular, as illustrated in FIG. 1B, brain tissue 62, such as obex tissue 64 and/or caudal medulla tissue 66 of the cervid, sheep or goat may be obtained for use in the present invention, as it has been demonstrated that these tissues contain PrP^{res} or another molecular alteration indicative of a TSE infection. As such, spinal cord tissue and/or spinal fluid may also be used. Consistent with the broader aspects of the invention, suitable tissue samples include additional organ tissues, including but not limited to eye, pancreatic tissue, muscle tissue, tongue tissue, nerve cells and/or bone marrow. In particular, any cervid, sheep or goat tissue demonstrated to contain PrP^{res} or another TSE-indicating molecular alteration may be utilized in the methods of the present invention. As described herein, a “sample” refers to a tissue sample, blood and/or a biological fluid sample as previously described.

As described herein “untreated” samples include samples involving substantially no work-up (chemical, physical or mechanical manipulation) prior to testing. Untreated samples can include fresh and/or thawed samples and also include raw and/or unadulterated samples provided for testing involving substantially no sample preparation other than obtaining the proper amount of the sample. In particular, as distinguishable from the prior art, untreated samples are not homogenized or mixed with water, subjected to enzyme action or chemically or physically extracted.

Preferably, samples are utilized untreated and are neither frozen, heated, nor dried prior to testing. However, raw, untreated samples may be frozen at conventional storage temperatures, e.g. −35° C. or −70° C., or as required by the particular laboratory or testing facility. Frozen samples are cut and/or thinly sectioned to expose a new surface to the spectrometric test instrumentation prior to testing.

In preparation for testing, thin sample sections of approximately about 1 cm×about 1 cm×about 0.2 cm in dimension are obtained from the animal species. Sample sections of up to about 3 cm×about 3 cm×about 0.5 cm may also be obtained, depending on the particular requirements of the spectrometric instrumentation and testing accessories employed in performing the methods of the present invention. Where biological fluids are used, samples from approximately about 10 μl to about 1 ml are obtained from the subject. As will be readily apparent to those skilled in the art, sample size is dependent on not only specific test instrumentation but also on the availability of the sample and the need for further testing of the sample.

Any of the aforementioned sample types may be used to develop a calibration model for a particular animal species and can include several different breeds/varieties of that species, including, but not limited to, a deer calibration model (including red deer, white-tailed deer, mule deer, etc.), an elk calibration model, a sheep calibration model (including Dorset, Merino, Cotswold, Suffolk etc.) or a goat calibration model (including Rock Alpine, Toggenburg, Saanen, etc.) in order to classify unknown samples from the same type of animal species as TSE-positive or TSE-negative. In addition, a single calibration model can be developed for specific breeds of the same species of deer, elk, sheep or goats (e.g. a white-tailed deer calibration model/diagnostic test). Calibration models can also include models directed to a single gender. Thus, it will be apparent to those skilled in the art that the present methodology is unlimited as to either animal genotype (deer, elk, sheep or goat) or strain of TSE.

The type of unknown sample tested using the methodology of the present invention (e.g. lymph tissue, blood or brain tissue) is preferably the same type of sample used for developing the calibration model for the particular species. In addition, it is preferred that each sample used in a particular calibration model or used for a particular diagnostic test be stored at similar temperatures. For example, calibration data sets developed with samples stored at about −70° C. prior to testing should be used to test unknown samples stored at about −70° C. However, samples stored at, for instance, −35° C. and fresh samples may be used to develop the same calibration model—which can be used to test unknown samples stored at −35° C. and fresh samples.

The raw, untreated samples are placed directly on the optical bench and a complete scan of the sample is rapidly obtained. Because sample preparation is minimal, samples tested by the present method can be subsequently analyzed and/or confirmed by immunohistochemistry (IHC), Western Blot or another validated gold standard test for detecting CWD and/or scrapie.

Development of a Calibration Model.

To develop a calibration model, a calibration set of infrared absorption data (or alternatively reflectance data) is obtained from samples extracted from subjects that have previously been identified as TSE-positive or TSE-negative (for CWD or scrapie). The infrared absorbance spectra for each of the known samples are obtained at wavenumbers from about 7400 cm⁻¹ to about 350 cm⁻¹, preferably from about 4500 cm⁻¹ to about 350 cm⁻¹, and more preferably from about 4000 cm⁻¹ to about 500 cm⁻¹. Without limitation, a more discreet wavenumber range from about 1700 cm⁻¹ to about 1600 cm⁻¹ may also be used to develop a calibration model—wherein such a wavenumber region includes at least a portion of the spectral changes observed between TSE-positive and TSE-negative samples. Thus, consistent with the broader aspects of the present invention, absorbance spectra may be obtained at any range of wavenumbers or wavelengths, or may be obtained in a more discrete range of wavenumbers or wavelengths, depending on the type of sample, the spectrometric method used, and/or the particular TSE to be diagnosed.

The calibration/reference set of infrared absorption data can be prepared from any number of reference samples, however, the calibration/reference data set will preferably include greater than about 10 reference samples.

Each sample in the calibration set can be scanned multiple times and co-added to increase the signal to noise ratio, thereby enhancing the spectral absorbance features of the sample (that is, the spectral features correlated in the frequency/wavelength domain). For example, tissue samples may be scanned approximately about 64 times, while biological fluids may be scanned approximately about 16 to about 32 times. One skilled in the art will appreciate that a sample may be scanned as many times as necessary to sufficiently distinguish/enhance the correlated spectral features from spectral noise (which shows no correlation in the frequency/wavelength domain). Generally, the more spectra included in the calibration set, the better chance there is of accounting for sample variations/impurities, instrument drifts and changes, and/or operator mistakes; thus, the test will be more sensitive. Irrespective of the number of multiple scans collected for a given sample, it is contemplated by the present invention that complete scanning of a single sample (including multiple scans of the same sample) can be performed, preferably, in less than about one minute.

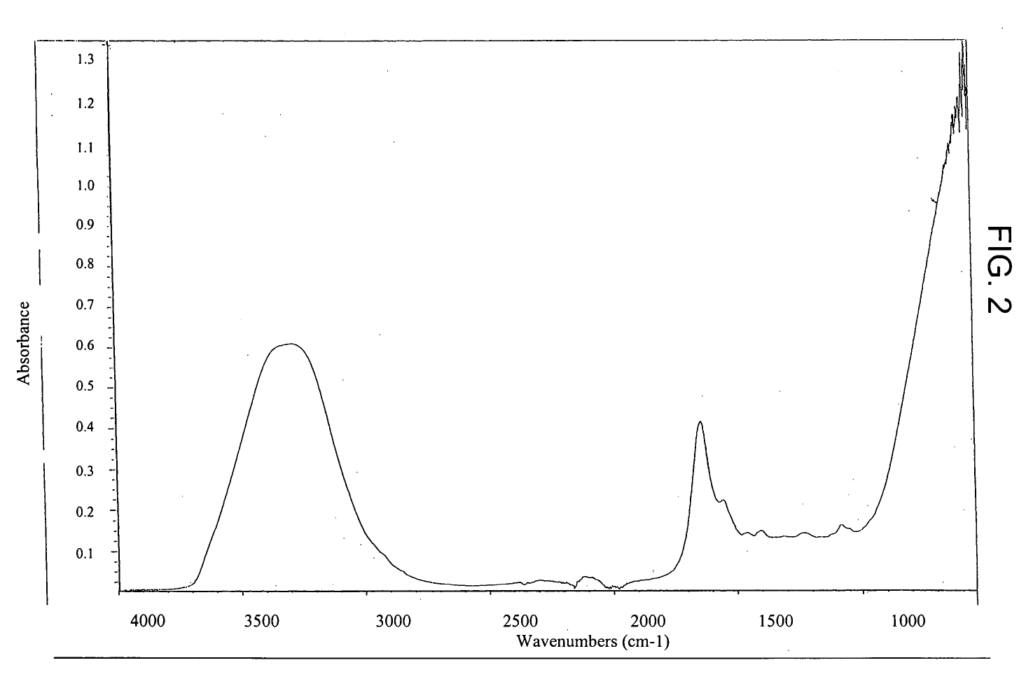

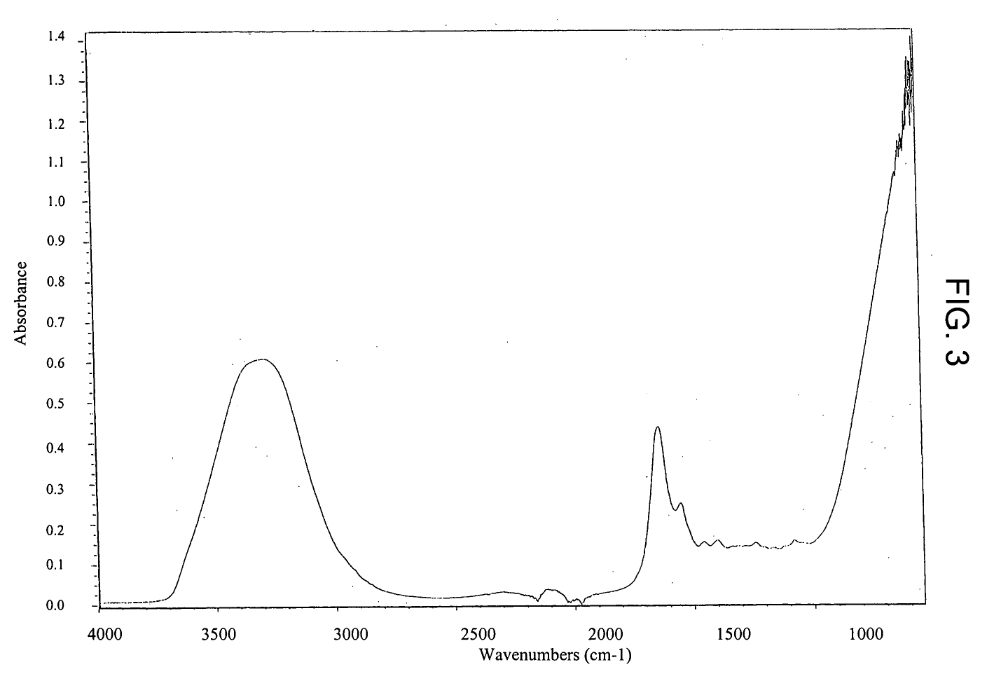

After each calibration/reference sample is scanned and the respective spectra collected, corresponding calibration models characteristic of a TSE-positive sample and a TSE-negative sample are developed from the calibration spectral data. FIG. 2 is an example of raw spectral data for a lymph tissue sample taken from a known positive (CWD-infected) white tailed deer subject and FIG. 3 is an example of absorbance spectra for a lymph tissue sample taken from a known negative (disease free) white tailed deer subject.

Preferably, prior to performing any of the data analysis methods described herein, original spectra is converted to second derivatives to enhance spectral details, such as shoulders of IR bands and to minimize or eliminate baseline shifts. However, the original spectra and/or first derivatives can be used in the spectral analysis for development of the predictive model, as will be well known to those skilled in the art. Data smoothing may be performed using a range of Savitzky-Golay algorithms or by weighted averaging the data using a set of weights that are a normalized Gaussian distribution. Further, any other data smoothing technique known to those skilled in the art may be used.

It will also be readily apparent to those skilled in the art that the data may first be mean centered, linearized, or otherwise normalized before such data analysis. Normalization of spectra may also be required when the calibration data/diagnostic test is transferred to a different spectrometric instrument (i.e. a different testing location), or where samples are tested on different spectrometric instruments. For example, Multiplicative Scatter Correction to account for pathlength variation can be performed prior to utilizing one of the data analysis methods referenced herein.

In order to reduce the amount of spectral data for analysis, the original spectra obtained for each of the known TSE-positive calibration samples are averaged and the original spectral obtained for each of the known TSE-negative samples are averaged. The average spectra for the TSE-positive samples and the average spectra for the TSE-negative samples are subtracted and/or the average spectra are superimposed in a manner that highlights the wavenumber regions exhibiting the most prominent spectral distinctions between the average TSE-positive and the average TSE-negative spectra. These spectral regions are then selected for further analysis/discrimination between the spectra of TSE-positive and TSE-negative samples. Consistent with the broader aspects of the present invention, data reduction can be performed on original (raw) spectra, first or second derivative spectra, data smoothed and/or normalized spectra.

In addition, it will be apparent to those skilled in the art that the entire spectral range of wavenumbers obtained during scanning of the calibration samples can be used in the methodology of the present invention, without utilizing a data reduction technique, provided the data analysis method utilized is capable of sufficiently discriminating between TSE-positive and TSE-negative samples in the calibration set such that unknown TSE-positive and TSE-negative samples can be accurately diagnosed.

Preferably, the method utilizes principal component analysis (PCA) as a chemometric method for breaking down spectroscopic data into its most basic variations. This type of multivariate technique analyzes entire regions of a spectrum and allows discrimination between the spectra of different groups of samples. In particular, PCA data transformation converts a set of correlated spectral features/variables (the raw spectral data) into a compressed smaller set of uncorrelated variables. This transformation rotates the coordinate system, resulting in the alignment of information on a fewer number of axes than in the original spectral arrangement. This results in a compression of the variables by allowing those variables that are highly correlated with one another to be treated as a single entity. After using PCA, there will be a small set of uncorrelated variables representing most of the information that was in the original set of variables; however, the information will be easier to use in subsequent analytical models.

Consistent with the broader aspects of the invention, any mathematical data analysis technique, spectral data compression technique, predictive modeling method, and/or chemometric analysis method known to those skilled in the art can be used to identify spectral correlation corresponding to a TSE-positive and a TSE-negative disease state within the calibration data set, provided the data analysis technique is capable of sufficiently discriminating between TSE-positive and TSE-negative samples in the calibration set such that unknown samples can be accurately diagnosed. Such techniques can include but are not limited to, Partial Least Squares Regression (PLS), Principal Component Regression (PCR), Multiple Linear Regression (MLR) and Discriminant Analysis. It will at once be appreciated by those skilled in the art that any of the data smoothing, normalization, statistical methods and/or data analysis techniques described herein can be performed using a commercially-available computer software program. Such programs can include, but are not limited to OMNI® (2002 Thermo Electron Corporation) including TQ Analyst® software (from Thermo Electron Corporation, Madison, Wis., U.S.A.); or PLSplusIQ™ and GRAMS/AI™ (from Thermo Galactic, Salem, N.H., U.S.A).

Accordingly, principal component analysis identifies patterns in the calibration/reference set spectra that contribute the most to the variation among that group, resulting in mathematical spectra (called loading vectors or principal components) which represent the most common variations to all the calibration data. The first principal component is constructed to account for as much variability as possible; this often corresponds to the major visual differences among spectra as caused by light scatter, path length changes, or particle size differences. The second principal component is constructed to account for as much of the remaining variability as possible. This often corresponds to the largest absorbance band changes, such as moisture. Additional components will include variation in other bands. A discussion of the underlying theory and calculations of principal component analysis can be found, for example, in Fredericks, et al., Applied Spectroscopy, 39, 303 (1985); Aries, et al., Spectroscopy, 5, 41 (1990); and Haaland, et al., Analytical Chemistry, 60, 1193 (1988).

The amount of variability accounted for in each principal component depends on the specific TSE, the type of sample, and the number of samples in the calibration set. Each time a calibration set is developed, optimized or expanded, the number of principal components used for the predictive model can fluctuate, as needed, to account for variability approaching about 100%. Thus, in order to develop a sensitive calibration model, the number principal components used may account for as much variability in the calibration spectra as possible and/or as practical. Typically, about 2 to about 25 principal components will account for about 85% to greater than about 99.9% of the variance of the variables. However, up to about 50 or more principal components can be used, depending on the type of sample used and the variability thereof.

The principal components are then used to predict the disease-state of “unknown” samples. To do this, a set of scaling coefficients called principal component scores (“PC scores”), for each principal component, are calculated for each sample in the calibration set. When the PC scores are multiplied by the principal components, and the results are summed, the original spectra are reconstructed. By knowing the set of principal components, the PC scores will represent the spectra as accurately as the original spectral responses at all the wavelengths considered. Thus, scatter plots of the PC scores over the principal components can provide a means for visualizing and summarizing the spectral data.

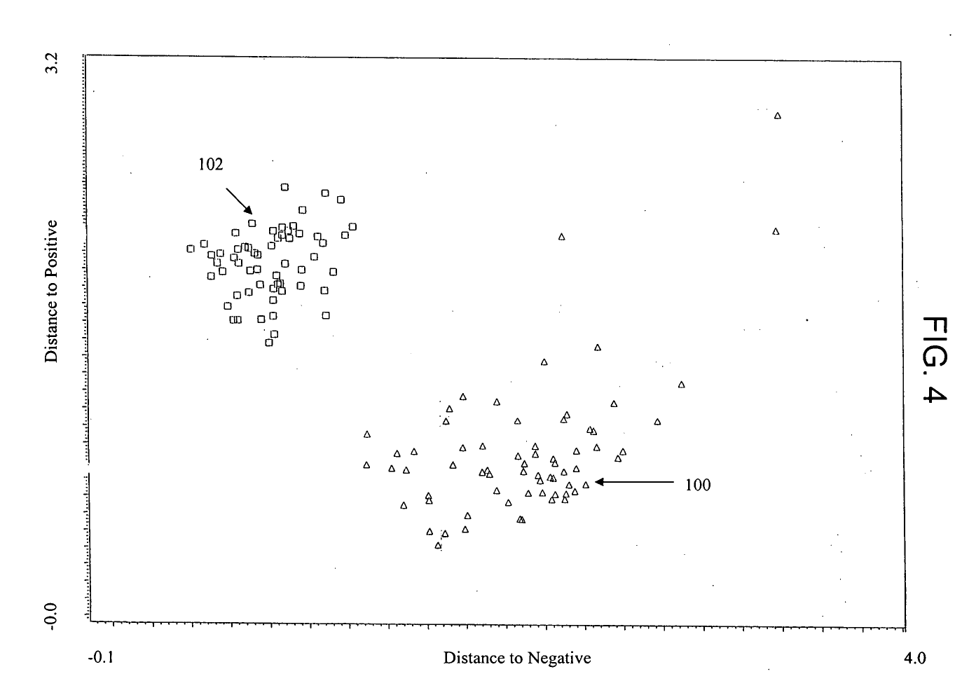

In a first example of the present invention, principal component analysis showed that the first ten (10) principal components accounted for about 98% of the total variance of the calibration tissue spectra. As illustrated in FIG. 4, a scatter plot of the calibration spectral data set after PCA transformation illustrates two distinct clusters of samples, a first cluster 100 corresponding to CWD-positive samples (triangular data points) and a second cluster 102 corresponding to CWD-negative samples (square data points). Accordingly, the calibration data set is used, after PCA transformation, to differentially identify TSE-positive and TSE-negative samples by comparing the unknown sample's spectral PC scores to the PC scores determined from the spectral data of known samples in the calibration set.

After all the calibration samples are scanned and the PC scores for each calibration sample in the calibration data set are calculated, the mean center PC scores are calculated for both the TSE-positive and TSE-negative cluster groupings. Alternatively, a mean centered spectra can be calculated for the entire calibration data set by averaging the absorbance/reflectivity values of the calibration samples at each wavelength to determine a mean spectra for each of the TSE-positive and TSE-negative cluster groupings, and the mean centered PC scores can be calculated therefrom. The mean center PC scores will be unique to each specific cluster grouping.

Further, the calibration set can be cross-validated by calculating the Mahalanobis distances from each known sample in the calibration set to the mean center TSE-positive PC scores and the mean center TSE-negative PC scores of the remaining known samples in the calibration set (e.g. by rotating each cross-validated sample out of the calibration set and calculating the Mahalanobis distances for that sample from the mean center TSE-positive PC scores and the mean center TSE-negative PC scores of the remaining known samples in the calibration set.) As well known to those skilled in the art, the Mahalanobis distance is used for determining the “similarity” of a sample to a reference set, for example, by comparing the unknown sample's PC scores to the mean center PC scores of both the TSE-positive and the TSE-negative cluster groupings of the calibration data set.

To determine whether an unknown sample is TSE-positive or TSE-negative, the unknown sample (or, alternatively, a validation sample previously identified as TSE-positive or TSE-negative) is first scanned to obtain the sample's spectral information. The PC scores of the unknown sample are then calculated. The Mahalanobis distances from both the mean center of the TSE-positive cluster grouping and the TSE-negative cluster groupings for the particular unknown sample are calculated from the calculated PC scores for the unknown sample. If the Mahalanobis distance from the unknown sample's PC scores to the mean center TSE-positive PC scores is closer than the Mahalanobis distance from the unknown sample's PC scores to the mean center TSE-negative PC scores, the unknown sample is classified as TSE-positive. Likewise, if the Mahalanobis distance from the unknown sample's PC scores to the mean center TSE-negative PC scores is closer than the Mahalanobis distance from the unknown sample's PC scores to the mean center TSE-positive PC scores, the unknown sample is classified as TSE-negative.

If both Mahalanobis distances (to the mean center TSE-negative PC scores and to the mean center TSE-positive PC scores) calculated during cross-validation of the calibration samples, during a validation sample test or during an unknown sample test are greater than 3, the sample is considered a Mahalanobis outlier. Such Mahalanobis outliers are considered a <No Test> result. Mahalanobis outliers may be caused by improper sample trimming techniques or contamination/corruption of the tissue sample and may not result in a proper diagnosis. Thus, any samples tested via the method of the present invention that are Mahalanobis outliers can be removed from the calibration data set, and if the sample is a validation sample, will result in a <No Test> result.

The method of the present invention also includes updating the calibration data set for the particular animal species (deer, elk, sheep or goat) with correctly diagnosed “unknown” sample spectra in order to continually update the calibration set. This permits the calibration model to best account for possible variation, such as strain differentiation, typically found in naturally occurring CWD or scrapie infections. As such, the calibration set for a given species can be continually optimized using samples from the same species that have been identified as TSE-positive and TSE-negative (and, if necessary, verified using another conventionally known TSE testing method). In addition, calibration data sets can be developed and/or optimized not only for a particular species, but also for a particular geographical area, region or state, or a specific chemical, physiological or physical characteristic of a TSE.

In addition, the methods of the present invention include use of the validated diagnostic test/predictive model at several locations or laboratories in order to provide remote testing capabilities permitting researchers, farmers, and/or breeders the ability to correctly and rapidly diagnose an animal with a TSE.

Sample Scoring.

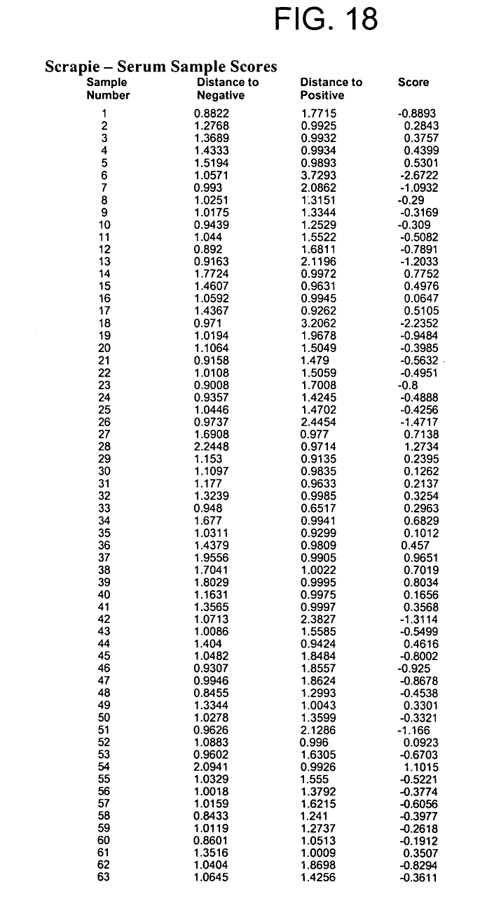

Samples tested in accordance with the present invention are scored with respect to a TSE-negative or TSE-positive result. Sample scores are calculated from the Mahalanobis distances determined from the unknown sample PC scores to the mean center of the TSE-positive cluster grouping and the mean center TSE-negative cluster groupings. The score is calculated by subtracting the (Mahalanobis) distance to positive from the (Mahalanobis) distance to negative. (Reference is made to Example 5B and FIG. 18).

Any TSE-negative sample, as determined by the calibration model, having a calculated score ranging between about −0.1000 to 0.0000 may be classified as <suspect positive>. For example a calculated score of −0.0900 would be classified as a <suspect positive> sample. Preferably, <suspect positive> samples are further tested by another TSE testing method such as by Western Blot assay.

In general, sample scoring permits the diagnosis of the sample to be quantified, Thus, depending on the particular animal genotype or TSE strain, calculation of the sample score may be adjusted to increase the sensitivity of the test, perhaps, at the expense of specificity of the test. For example, sample scoring calculations may be adjusted to be more sensitive to, and therefore increase the incidence of, a <suspect positive> result, ensuring the number of false negative results of the test are substantially zero.

After testing is completed on a sample, or alternatively, a group of samples, diagnosis reports can be generated. Such reports can contain animal information, the origin of the animal, animal owner, diagnosis, lot information, etc. in order to provide researchers or governments with information for tracking and monitoring the animal and/or animal byproducts.

Thus, with reference to the preceding, the present invention includes, in part, an infrared spectroscopic method for developing a reference or calibration model for use in differentially diagnosing naturally occurring chronic wasting disease and/or scrapie, comprising (1) obtaining a plurality of known TSE-positive and known TSE-negative, untreated tissue samples from a given species to form a calibration set; (2) analyzing each sample in the calibration set using an IR spectrometric method and obtaining calibration IR spectral data for each known tissue sample in the calibration set; (3) applying a multivariate chemometric technique to the calibration IR spectral data for each sample to statistically differentiate the TSE-positive and TSE-negative calibration IR spectral data contained in the calibration set. The method can also include updating the calibration set with IR spectra of similar tissue samples from the same animal species and/or variety/breed that have been correctly diagnosed using the method of the present invention and verified via another TSE diagnostic technique. The method can include using tissue samples from the cortex or paracortex of a lymph node from the species, obex tissue from the brain of the animal and/or utilizing a blood sample from the animal. Preferably, the sample is irradiated at wavenumbers from about 4500 cm⁻¹ to about 350 cm⁻¹.

In part, the present invention can include a method for detecting a transmissible spongiform encephalopathy in an animal selected from the group consisting of deer, elk, sheep and goat comprising selecting a sample from a live or postmortem subject to determine whether the subject has a transmissible spongiform encephalopathy; obtaining IR spectral data from the sample; and determining whether the sample is TSE-positive or TSE-negative by differentially comparing the IR spectral data of the sample with a calibration model comprising calibration IR spectral data from a plurality of known TSE-positive and known TSE-negative samples of similar type from the same species as the subject.

Accordingly, in part the present invention also includes a method for detecting a TSE specific to a cervid, sheep or goat. The method comprises (1) obtaining a sample from one of a cervid, sheep or goat, (2) directing a beam of IR light at wavenumbers from about 4500 cm⁻¹ to about 350 cm⁻¹ to produce IR spectral data for the sample, (3) comparing the IR spectral data for the sample with a calibration set of IR spectral data obtained through multivariate analysis of known TSE-positive and known TSE-negative samples, and (4) determining whether the sample is TSE-positive or TSE-negative from the calibration data set.

In part, the present invention is a method of detecting a change in spectral response between diseased and normal samples to differentially diagnose and/or detect Chronic Wasting Disease. The method comprises (1) obtaining a sample from at least one of a lymph node containing cortex or paracortex tissue, obex tissue or blood from a cervid (2) directing a beam of IR light at wavenumbers from about 4500 cm⁻¹ to about 350 cm⁻¹ to produce IR spectral data for the sample, (3) comparing the IR spectral data for the sample with a calibration set of infrared spectral data comprising both a CWD-positive and CWD-negative predictive model, and (4) determining whether variation in infrared absorption occurs in the sample, within at least one range of wavenumbers, due to the variation being characteristic of a positive indicator of CWD or a negative indicator of CWD.

The present invention also includes detecting a change in spectral response between diseased and normal samples to differentially diagnose and/or detect scrapie. The method comprises (1) obtaining a sample from at least one of a lymph node containing cortex or paracortex tissue, obex tissue or blood from a sheep or a goat species, (2) directing a beam of IR light at wavenumbers from about 4500 cm⁻¹ to about 350 cm⁻¹ to produce IR spectral data for the sample, (3) comparing the IR spectral data for the sample with a calibration set of infrared spectral data comprising both a scrapie-positive and scrapie-negative predictive model, and (4) determining whether variation in infrared absorption occurs in the sample, within at least one range of wavenumbers, due to the variation being characteristic of a positive indicator of scrapie or a negative indicator of scrapie.

In part, the present invention is a method for rapidly screening samples for Chronic Wasting Disease or scrapie. The method comprises (1) analyzing an untreated cortex or paracortex sample from the lymph node, untreated obex tissue or untreated blood from a cervid (if screening for CWD) or a sheep or goat species (if screening for scrapie) using an IR spectroscopic method providing IR spectral data, (2) applying principal component analysis to the IR spectral data obtained from the sample and calculating the PCA scores for the unknown sample, and (3) differentially determining whether the sample is positive or negative for the TSE by comparing the PC scores for the sample with a calibration IR spectral data set comprising mean centered PC scores for the TSE-positive and TSE-negative samples in the calibration IR spectral data set. The method can further include confirming of a positive CWD (or scrapie) diagnosis using a validated assay method such as immunohistochemistry.

Differentially determining whether the unknown sample is TSE-positive or TSE-negative can include calculation of the Mahalanobis distance from the unknown sample's PC scores to the mean centered PC scores for the TSE-positive and TSE-negative sample groupings in the calibration spectral data set and determining whether the Mahalanobis distance from the unknown sample's PC scores is closer to the mean center TSE-positive PC scores or closer to the mean center TSE-negative PC scores.

Accordingly, it can be that in part, the present invention is a method for monitoring chronic wasting disease and/or scrapie occurring within a particular region, state or country. The method comprises (1) presenting a hunter harvested, naturally postmortem or live animal species, (2) recording the originating location of the animal, (3) obtaining an untreated sample from the animal, the tissue sample selected from cortex or paracortex sample from the lymph node of the animal, brain tissue or blood, (4) labeling the sample with computer readable indicia (such as a bar code) which includes but is not limited to the location of the animal, hunter/farmer information, and/or the date, (5) obtaining absorbance or emission spectral data for the sample at wavenumbers from about 7400 cm⁻¹ to about 350 cm⁻¹, (6) comparing the spectral data to a reference set of spectral data, (7) differentially identifying the sample as either positive or negative as indicated by the reference set of spectral data.

Moreover, the present invention includes, in part, a method of using principal component analysis to discriminate between IR spectra of TSE-positive and TSE-negative samples. The method consists essentially of obtaining calibration IR absorbance spectra for a plurality of untreated known TSE-positive and untreated known TSE negative samples, applying principal component analysis to the calibration IR absorbance spectra obtained for each of the known TSE-positive and known TSE-negative samples to statistically discriminate between the calibration IR absorbance spectra of the known TSE-positive samples and the known TSE-negative samples, calculating the TSE-positive mean center principal component scores for the known TSE-positive samples and calculating the TSE-negative mean center principal component scores for the known TSE-negative samples, obtaining IR absorbance spectral data from an untreated, unknown sample, performing principal component analysis on the unknown sample including determining the principal component scores of the unknown sample and calculating a Mahalanobis distance from the principal component scores of the unknown sample to the TSE-positive mean center principal component scores and calculating a Mahalanobis distance from the principal component scores of the unknown sample to the TSE-negative mean center principal component scores; whereby the unknown sample is classified as TSE-positive or TSE-negative.

### EXAMPLES OF THE INVENTION

The following non-limiting examples and data illustrate various aspects and features of the present invention, including the surprising and unexpected results obtained thereby. It should, of course, be understood that these examples are included for illustrative purpose only and that the invention is not limited to the collection conditions, IR test instrumentation or the like set forth herein. Comparable utility and advantages can be realized using various other data analysis methodologies and/or embodiments consistent with the scope of this invention.

Test Equipment

All samples in the Examples that follow were scanned using a lab grade FTIR spectrometer (Nexus 670 by Thermo Nicolet, Inc., Madison, Wis.) including a standard mid-infrared source (7400-350 cm⁻¹, res. 0.125 cm⁻¹) and a DLaTGS detector (deuterated trigycine sulphate doped with L-alanine) with KBr window and was equipped with a DuraSamplIR II™ 3-Reflection Diamond/ZnSe (SensIR™ Technologies, Danbury, Conn.). However, any commercially available infrared spectrophotometer may be utilized. Data collection and manipulation software OMNIC (© 2002 Thermo Electron Corporation) including TQ Analyst software (© 2002 Thermo Electron Corporation), capable of performing principal component analysis, was included with the instrument and used to perform the statistical analysis. However, any computer program known to those skilled in the art may be used to carry out the statistical methods taught by the present invention.

### Example 1

### Development of a Calibration Model for Diagnosing CWD in Lymph Tissue

Tissue Handling

A retropharyngeal lymph node from each subject was sectioned to expose the cortex and paracortex of the node, as illustrated in FIG. 1A.

An approximately about 1 cm×about 1 cm×about 0.2 cm section of a lymph node from a subject was then collected from the cortex or paracortex of the lymph node to obtain sample sections containing primary and/or secondary follicles. Samples were trimmed, if required, to minimize medullary tissue within the sample. The exposed tissue was placed directly on the optical bench of the FTIR instrument without further work-up.

Sample Sources

With respect to the calibration model data, 29 known CWD positive and 34 known CWD negative samples were collected from white tailed deer in Nebraska and in Mississippi and were obtained from the U.S. Department of Agriculture/Agricultural Research Service in Pullman, Wash. An additional 38 known positive samples and 26 known negative samples, taken from white tailed deer harvested during the 2002 deer hunting season, were obtained from the State of Wisconsin Department of Natural Resources. In all samples, a CWD diagnosis was predetermined using standard gold standard methods such as by Western Blot assay or IHC.

The calibration data set, consisting of all 127 known samples, was used for spectral comparison via principal component analysis.

With respect to the validation data, 26 known positive samples and 59 known negative samples, taken from white tailed deer harvested during the 2002 deer hunting season, were obtained from the State of Wisconsin Department of Natural Resources. In all 85 samples, a CWD diagnosis was predetermined using IHC. The validation samples were obtained from different subjects than the samples used for the calibration set.

Testing

Each sample in the calibration and validation sets was scanned 64 times and co-added to increase the signal to noise ratio, thereby enhancing the spectral absorbance features of the sample. Complete scanning of each sample (including the 64 scans of the same sample) was performed in less than about one minute. The FTIR data collection software, OMNIC/TQ Analyst (© 2002 Thermo Electron Corporation), collected the dispersive spectra for each sample. FIGS. 2 and 3 are representative of the raw spectral data obtained for a CWD-positive sample (FIG. 2) and a CWD-negative sample (FIG. 3).

After scanning, each tissue sample was returned to its sample container for subsequent analysis by other methods.

FTIR Calibration for Chronic Wasting Disease

Calibration of the test for CWD was performed using discriminate principal component analysis performed with the analysis software described herein. It will be appreciated by those skilled in the art that other multivariate chemometric techniques may be used for data analysis in developing the calibration/reference data set, including but not limited to, Partial Least Squares Regression (PLS), Principal Component Regression (PCR), Multilinear Regression Analysis (MLR) and Discriminant Analysis.

The software was calibrated with the known CWD-positive and known CWD-negative samples to develop a calibration model specific for CWD (and/or PrP^{res}) in lymphoid tissue taken from cervids. Principal component analysis was performed on the calibration spectral data over the range from about 4500 cm⁻¹ to about 350 cm⁻¹ for each tissue sample in the calibration data set. As illustrated in Table 1, ten (10) principal components, accounting for about 98% of the variance in the calibration spectral data set were used for the calibration model.

TABLE 1
PRINCIPAL COMPONENT ANALYSIS OF
CALIBRATION DATA SET

| PRINCIPAL | | CUMULATIVE |
|---|---|---|
| COMPONENT | % VARIANCE | VARIANCE |
| 1 | 58.989 | 58.989 |
| 2 | 19.922 | 78.912 |
| 3 | 9.077 | 87.988 |
| 4 | 4.490 | 92.479 |
| 5 | 2.481 | 94.960 |
| 6 | 1.172 | 96.132 |
| 7 | 0.760 | 96.891 |
| 8 | 0.451 | 97.342 |
| 9 | 0.355 | 97.697 |
| 10 | 0.273 | 97.970 |

A set of PC scores for each tissue sample in the calibration spectral data set was calculated. In addition, the mean center PC scores were generated for the known CWD-positive samples and the known CWD-negative samples.

The calibration set was cross-validated by calculating the Mahalanobis distances from each known sample in the calibration set to the mean center CWD-positive PC scores and the mean center CWD-negative PC scores of the remaining known samples in the calibration set (e.g. by rotating each cross-validated sample out of the calibration set and calculating the Mahalanobis distances for that sample from the mean center CWD-positive PC scores and the mean center CWD-negative PC scores of the remaining known samples in the calibration set.)

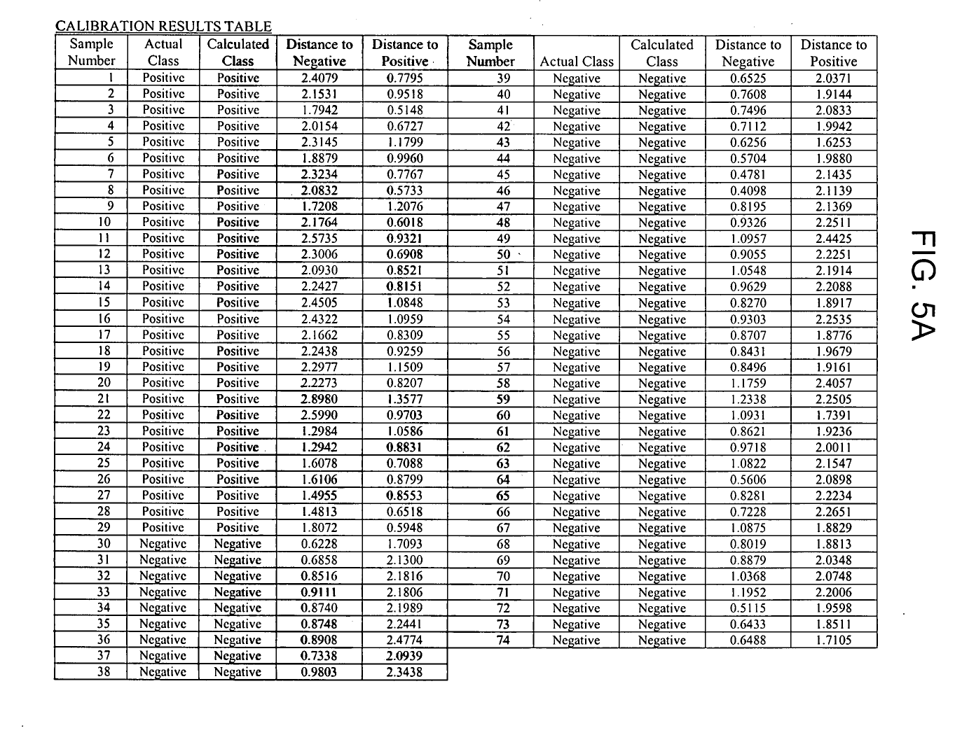

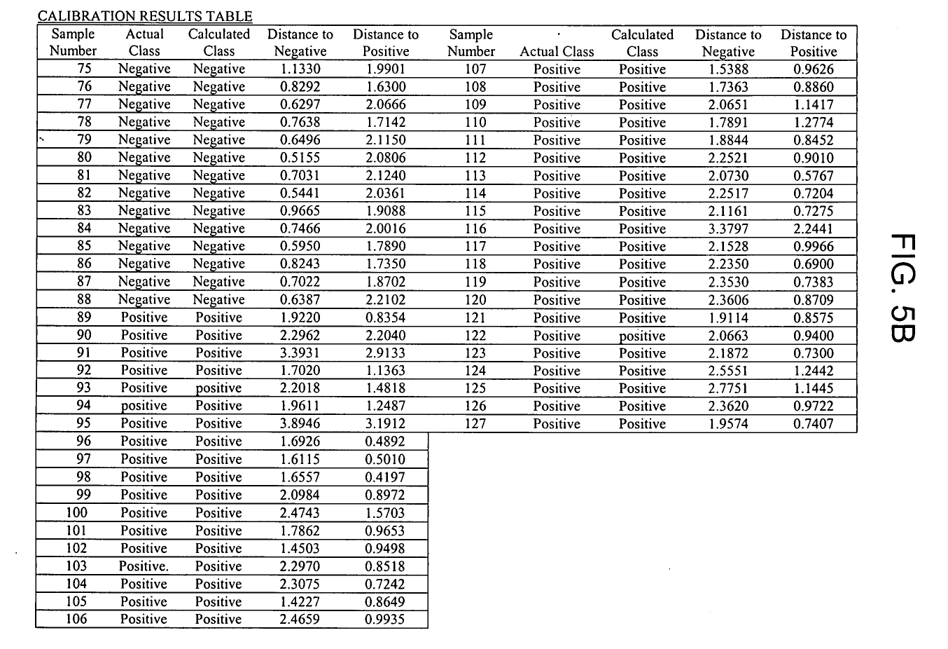

As illustrated in FIG. 4, a scatter plot of the Mahalanobis distances for each calibration sample spectra depicts two distinct clusters of calibration data points, a first cluster grouping 20 corresponding to CWD-positive tissue samples and a second cluster grouping 22 corresponding to CWD-negative tissue samples. FIGS. 5A and 5B contains a tabular representation of the data illustrating the actual class and the calculated class of the calibration data set. As illustrated in FIGS. 5A and 5B, all calibration samples were correctly cross-validated as either CWD-positive or CWD-negative.

Consistent with the broader aspects of the invention, correlation between the present testing methodology and a validated TSE test will be evaluated by the kappa calculation; and agreement of less than 96% will result in recalibration of the system using additional refinement of the calibration spectrum examined, in particular, the calibration will be adjusted so that the sensitivity of the test approaches 100%. A slight decrease in test specificity (resulting in a small increase in false positive samples) is preferred in a pre-clinical screening test for TSEs. All TSE-positive diagnosis can be confirmed by IHC or other validated diagnostic test.

Validation of the Calibration Set for the Method for Detecting CWD in Lymph Tissue

Analysis of Validation Spectral Data Using the Predictive Model

Principal component analysis was performed on the spectral data for each validation tissue sample using the ten principal components identified in the calibration model. These spectral features were analyzed by PCA and the PC scores for each validation sample were determined.

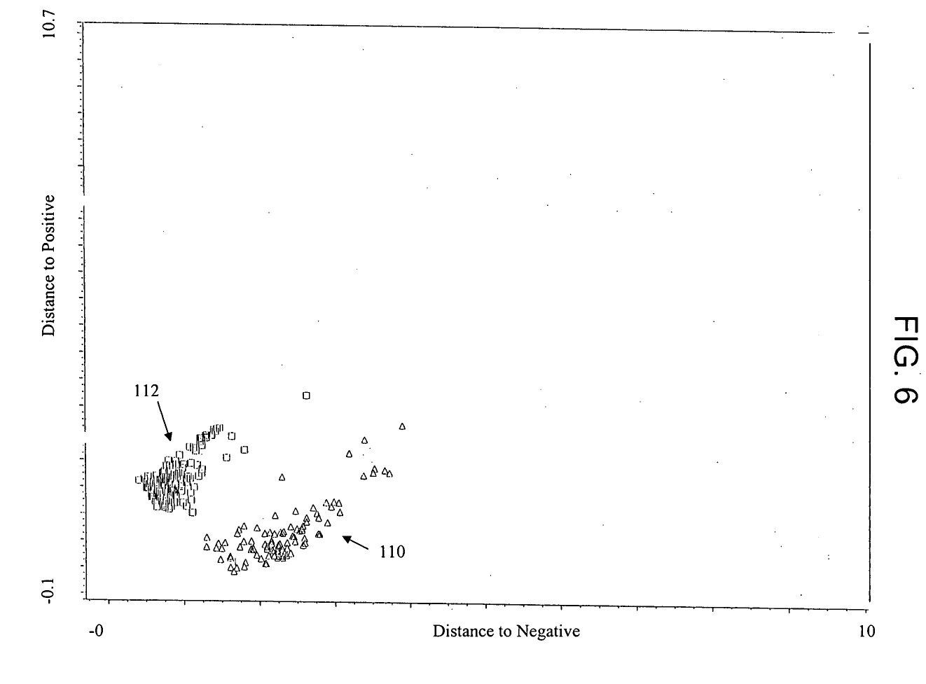

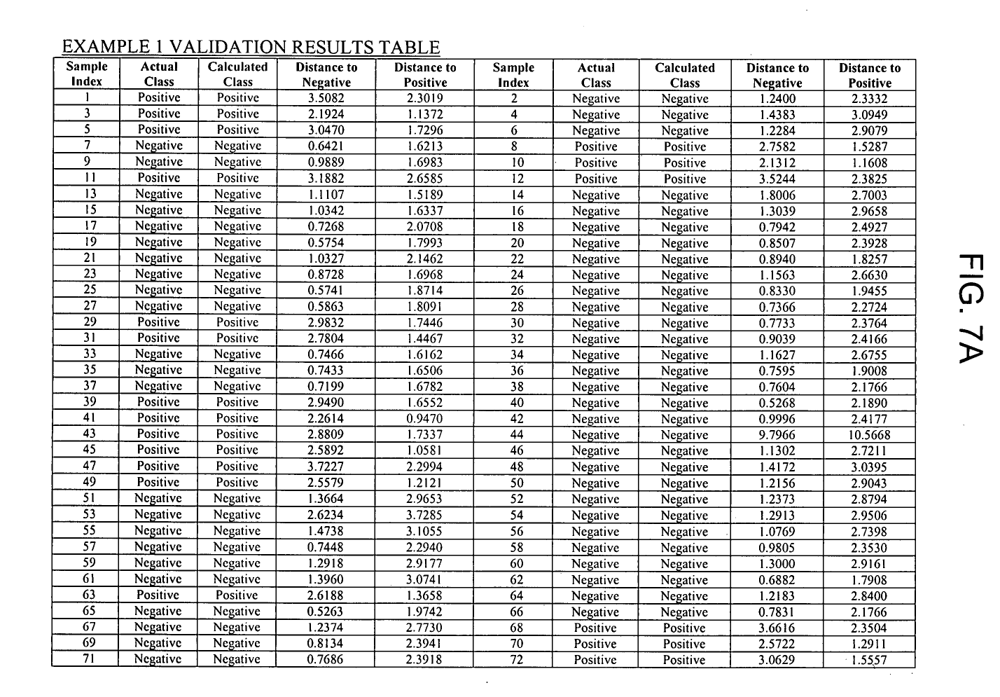

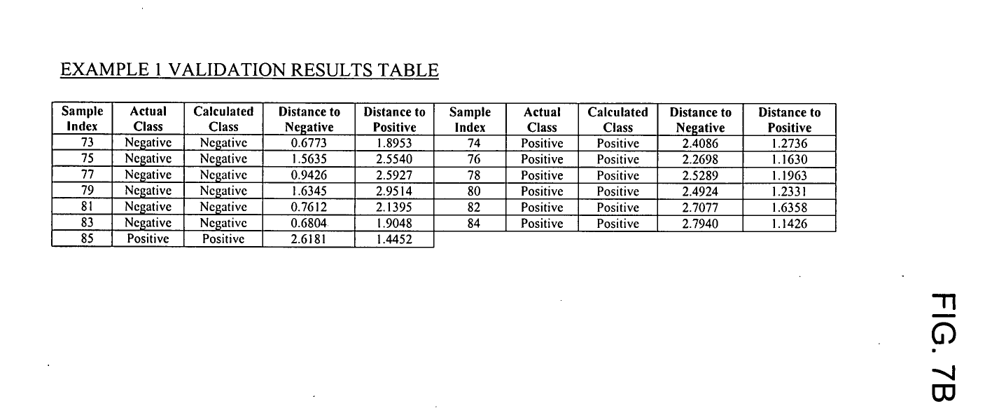

The Mahalanobis distance for each validation sample in the validation set was determined from the PC scores of the specific validation sample and the mean center PC scores of the CWD positive cluster grouping of the calibration data set. Likewise, the Mahalanobis distance for each validation sample in the validation set was determined from the PC scores of the validation sample and the mean center PC scores of the CWD negative cluster grouping of the calibration data set. If the Mahalanobis distance from the validation sample's PC scores to the mean center CWD positive PC scores was closer than the Mahalanobis distance from the validation sample's PC scores to the mean center CWD negative PC scores, the validation sample was classified as CWD positive. Likewise, if the Mahalanobis distance from the validation sample's PC scores to the mean center CWD negative PC scores was closer than the Mahalanobis distance from the validation sample's PC scores to the mean center CWD positive PC scores, the validation sample was classified as TSE negative. FIG. 6 illustrates a scatter plot of the Mahalanobis distances for each validation sample. As can be seen, the validation samples are aligned with the two distinct clusters of groups of the calibration data set, and thus, each sample corresponds to either a CWD-positive disease state (triangular points 110) or a CWD-negative disease state (square points 112). As illustrated in FIGS. 7A and 7B, each validation sample in the set of 85 samples were correctly diagnosed, thus, use of the present methodology revealed 100% agreement of the validation test results with the IHC results for each sample in the validation set.

### Example 2

### Optimization of the Calibration and Validation Sets for Diagnosing Chronic Wasting Disease of Example 1

Calibration of Method for Chronic Wasting Disease

The calibration data set of Example 1 was optimized by removing sample number 95 from the sample set. As illustrated in FIGS. 5A and 5B, this sample number resulted in a Mahalanobis distance of greater than 3. As such, the sample was considered a Mahalanobis outlier. According to the present methodology, such Mahalanobis outliers are considered a <No Test> result.

While sample number 95 was correctly cross-validated and diagnosed, as indicated in Example 1, it was removed from the calibration set for Example 2 in order to properly update the calibration model. Once removed from the calibration set, the principal components for the updated calibration model were recalculated as indicated below, and the Mahalanobis distances for each remaining sample were recalculated.

Ten principal components were used accounting for about 98% of the variability. These principal components are presented in Table 2.

TABLE 2
PRINCIPAL COMPONENT ANALYSIS OF OPTIMIZED
CALIBRATION DATA SET

| PRINCIPAL | | CUMULATIVE |
|---|---|---|
| COMPONENT | % VARIANCE | VARIANCE |
| 1 | 62.251 | 62.251 |
| 2 | 18.181 | 80.433 |
| 3 | 6.692 | 87.124 |
| 4 | 5.048 | 92.172 |
| 5 | 2.427 | 94.599 |
| 6 | 1.862 | 96.461 |
| 7 | 0.505 | 96.966 |
| 8 | 0.427 | 97.393 |
| 9 | 0.270 | 97.663 |
| 10 | 0.272 | 97.936 |

A set of PC scores for each tissue sample in the calibration spectral data set was calculated. In addition, the mean center PC scores were generated for the known CWD-positive samples and the known CWD-negative samples.

The calibration set was cross-validated by calculating the Mahalanobis distances from each known sample in the calibration set to the mean center CWD-positive PC scores and the mean center CWD-negative PC scores of the remaining known samples in the calibration set, as in Example 1.

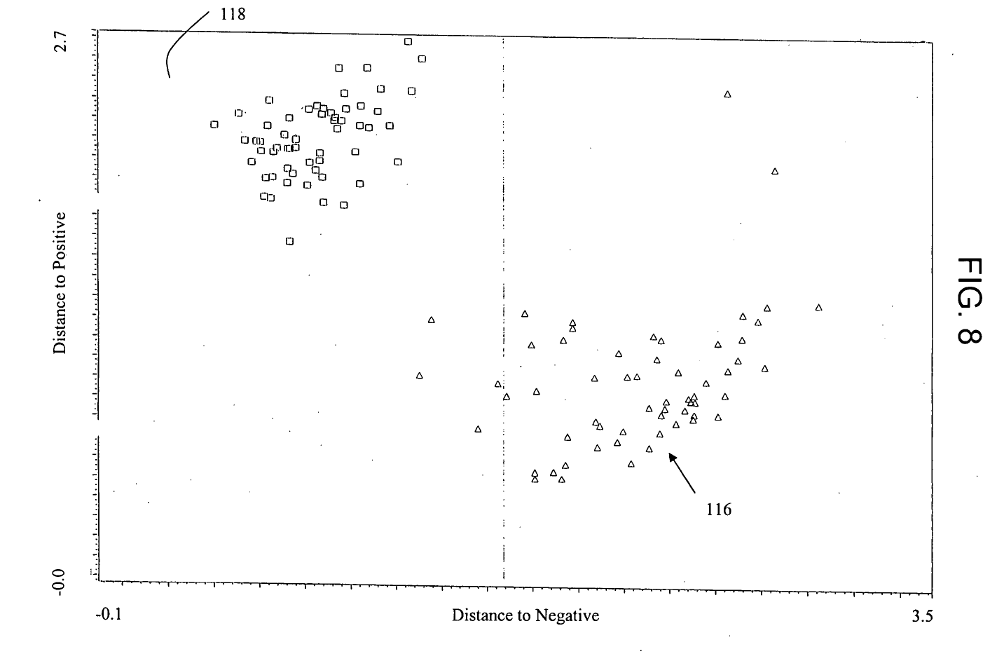

As illustrated in FIG. 8, a scatter plot of the Mahalanobis distances for each calibration sample spectra depicts two distinct clusters of calibration data points, a first cluster grouping 116 corresponding to CWD-positive tissue samples (triangular data points) and a second cluster grouping corresponding to CWD-negative tissue samples (square data points).

Validation of Method for Chronic Wasting Disease

As illustrated in FIGS. 7A and 7B, sample number 44 resulted in a Mahalanobis distance of greater than 3. As such, the sample was considered a Mahalanobis outlier. According to the present methodology, such Mahalanobis outliers are considered a <No Test> result.

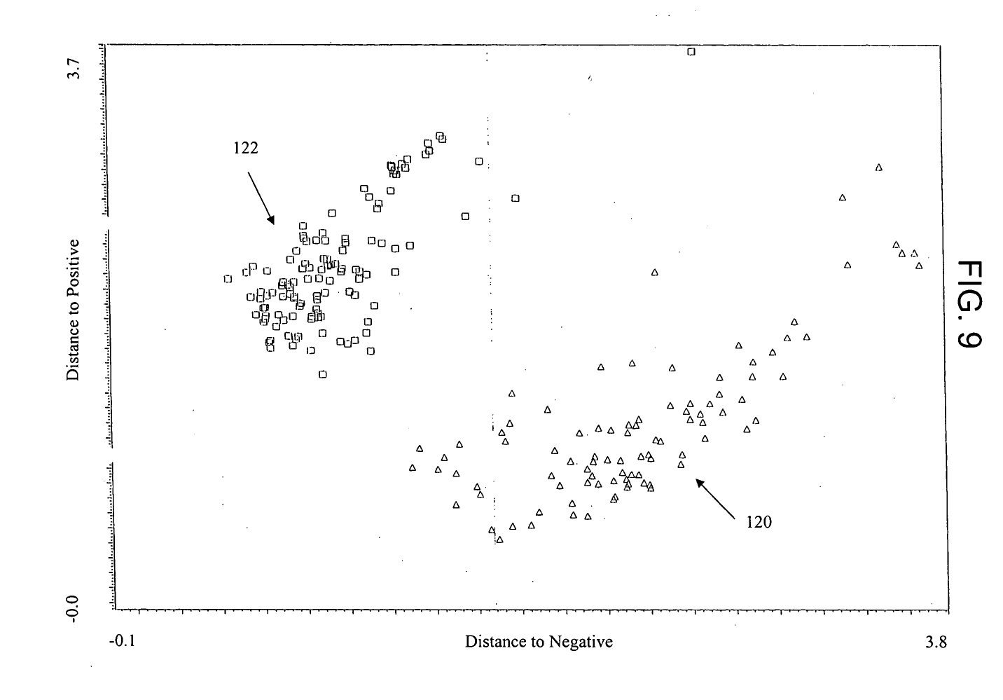

While sample number 44 was correctly cross-validated and diagnosed, as indicated in Example 1, it was removed from the validation set. The Mahalanobis distance for each validation sample in the validation set was determined from the PC scores of validation sample and the mean center PC scores of the CWD positive cluster grouping of the optimized calibration data set. Likewise, the Mahalanobis distance for each validation sample in the validation set was determined from the PC scores of validation sample and the mean center PC scores of the CWD negative cluster grouping of the optimized calibration data set. If the Mahalanobis distance from the validation sample PC scores to the mean center CWD positive PC scores was closer than the Mahalanobis distance from the validation sample PC scores to the mean center CWD negative PC scores, the validation sample was classified as CWD positive. Likewise, if the Mahalanobis distance from the validation sample PC scores to the mean center CWD negative PC scores was closer than the Mahalanobis distance from the validation sample PC scores to the mean center CWD positive PC scores, the validation sample was classified as TSE-negative. FIG. 9 illustrates a scatter plot of the Mahalanobis distances for each validation sample. As can be seen, the validation samples are aligned with the two distinct clusters of groups, corresponding to either a CWD-positive disease state (triangular data points 120) or a CWD-negative disease state (square data points 122).

As illustrated in FIG. 9, each validation sample in the set of 84 samples were correctly diagnosed, thus, use of the present methodology revealed 100% agreement of the validation test results with the IHC results for each sample in the validation set.

### Example 3

### Development of a Calibration Model for Diagnosing Scrapie in Lymph Tissue

Tissue Handling

A retropharyngeal lymph node from each subject was sectioned to expose the cortex and paracortex of the node, as illustrated in FIG. 1A.

An approximately about 1 cm×about 1 cm×about 0.2 cm section of a lymph node from a subject was then collected from the cortex or paracortex of the lymph node to obtain sample sections containing primary and/or secondary follicles. Samples were trimmed, if required, to minimize medullary tissue within the sample. The exposed tissue was placed directly on the optical bench of the FTIR instrument without further work-up.

Sample Sources

With respect to the calibration model data, 10 known samples positive for naturally occurring scrapie and 20 known negative samples were collected from sheep in the state(s) of Ohio, Montana, South Dakota, and Washington were provided by the U.S. Department of Agriculture/Agricultural Research Service in Pullman, Wash. In all samples, a scrapie diagnosis was predetermined using standard gold standard methods such as by Western Blot assay or IHC.

The calibration data set was then used for spectral comparison via principal component analysis that consisted of all 30 known samples.

With respect to the validation data, 5 known samples positive for naturally occurring scrapie and 10 known negative samples were obtained. The lymph nodes were sectioned as described previously herein. In all 15 samples, a scrapie diagnosis was predetermined using IHC. The validation samples were obtained from different subjects than the samples used for the calibration set.

Testing

Each sample in both the calibration set and the validation set were scanned 64 times and co-added to increase the signal to noise ratio, thereby enhancing the spectral absorbance features of the sample. Complete scanning of each sample (including the 64 scans of the same sample) was performed in less than about one minute. The FTIR data collection software, OMNIC/TQ Analyst (© 2002 Thermo Electron Corporation), collected the dispersive spectra for each sample.

FTIR Calibration for Scrapie in Sheep

Calibration of the test for scrapie was performed using discriminate principal component analysis performed with the analysis software described herein. As in Examples 1 and 2, it will be appreciated by those skilled in the art that other multivariate chemometric techniques may be used for data analysis in developing the calibration/reference data set, including but not limited to, Partial Least Squares Regression (PLS), Principal Component Regression (PCR), Multilinear Regression Analysis (MLR) and Discriminant Analysis.

The software was calibrated with the known scrapie-positive and scrapie-negative samples to develop a calibration model specific for scrapie infections in lymphoid tissue taken from sheep. Principal component analysis was performed on the calibration spectral data over the range from about 4500 cm⁻¹ to about 350 cm⁻¹ for each tissue sample in the calibration data set. As illustrated in Table 3, ten (10) principal components, accounting for over 98% of the variance in the calibration spectral data set were used for the calibration model.

TABLE 3
PRINCIPAL COMPONENT ANALYSIS
OF CALIBRATION DATA SET

| PRINCIPAL | | CUMULATIVE |
|---|---|---|
| COMPONENT | % VARIANCE | VARIANCE |
| 1 | 68.255 | 68.255 |
| 2 | 21.139 | 89.394 |
| 3 | 4.038 | 93.433 |
| 4 | 1.713 | 95.146 |
| 5 | 0.989 | 96.135 |
| 6 | 0.618 | 96.753 |
| 7 | 0.520 | 97.272 |
| 8 | 0.348 | 97.621 |
| 9 | 0.353 | 97.974 |
| 10 | 0.291 | 98.264 |

A set of PC scores for each tissue sample in the calibration spectral data set was calculated. In addition, the mean center PC scores were generated for the samples known to be positive for scrapie and the samples known to be negative.

The calibration set was cross-validated by calculating the Mahalanobis distances from each known sample in the calibration set to the mean center scrapie-positive PC scores and the mean center scrapie-negative PC scores of the remaining known samples in the calibration set (e.g. by rotating each cross-validated sample out of the calibration set and calculating the Mahalanobis distances for that sample from the mean center scrapie-positive PC scores and the mean center scrapie-negative PC scores of the remaining known samples in the calibration set.)

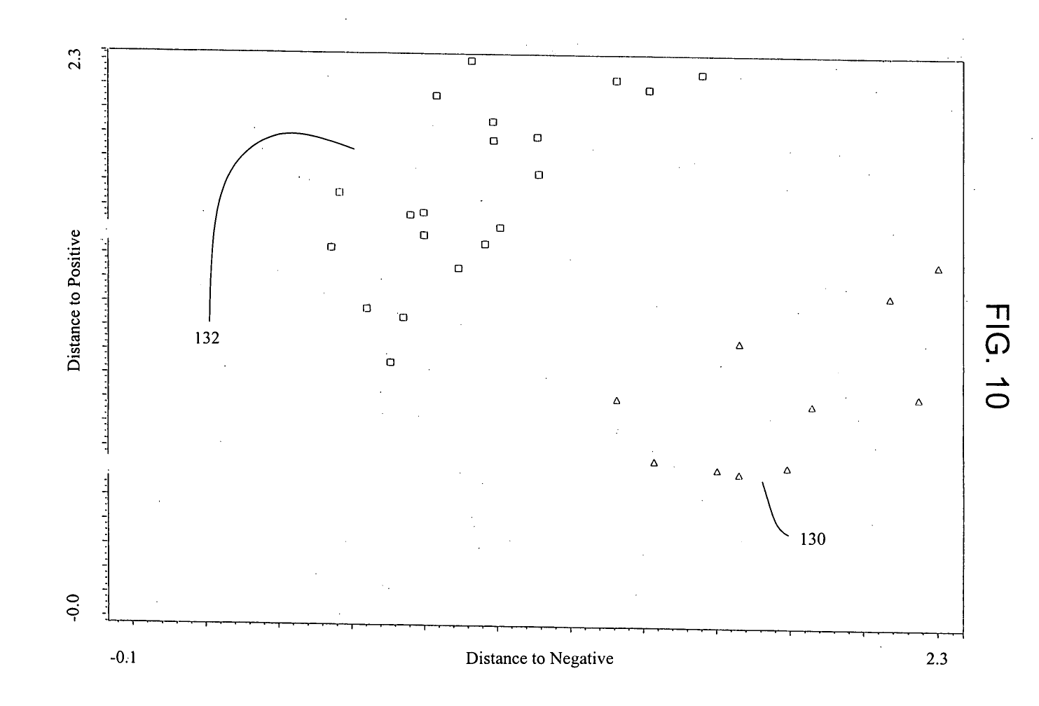

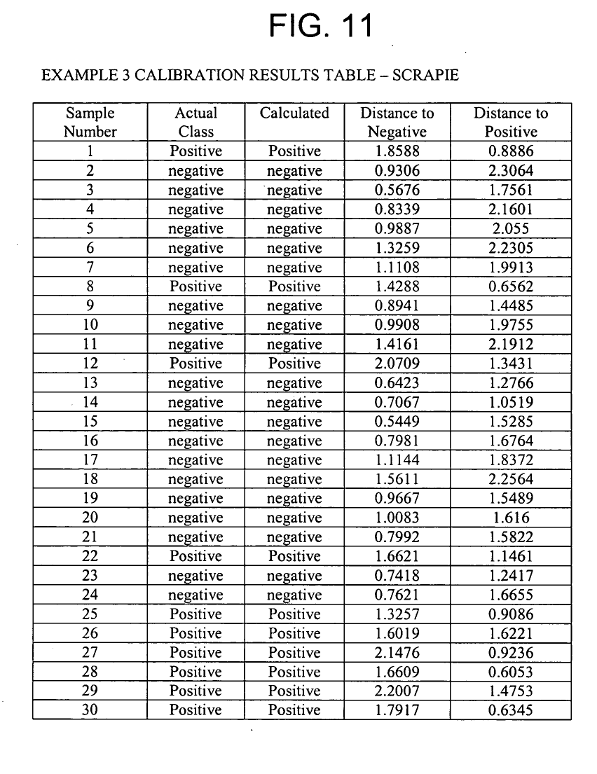

As illustrated in FIG. 10, a scatter plot of the Mahalanobis distances for each calibration sample spectra depicts two distinct clusters of calibration data points, a first cluster grouping corresponding 130 to scrapie-positive tissue samples (triangular data points) and a second cluster grouping corresponding 132 to scrapie-negative tissue samples (square data points). FIG. 11 contains a tabular representation of the calibration data samples illustrating the actual class and the calculated class for each sample of the calibration data set. As illustrated in FIG. 11, all calibration samples were correctly cross-validated as either scrapie-positive or scrapie-negative.

Validation of the Calibration Set for the Method for Detecting Scrapie in Lymph Tissue

Analysis of Validation Spectral Data Using the Predictive Model

Principal component analysis was then performed on the spectral data for each validation tissue sample using the ten principal components identified in the calibration model. These spectral features were analyzed by PCA and the PC scores for each validation sample were determined, as described with reference to Example 1.

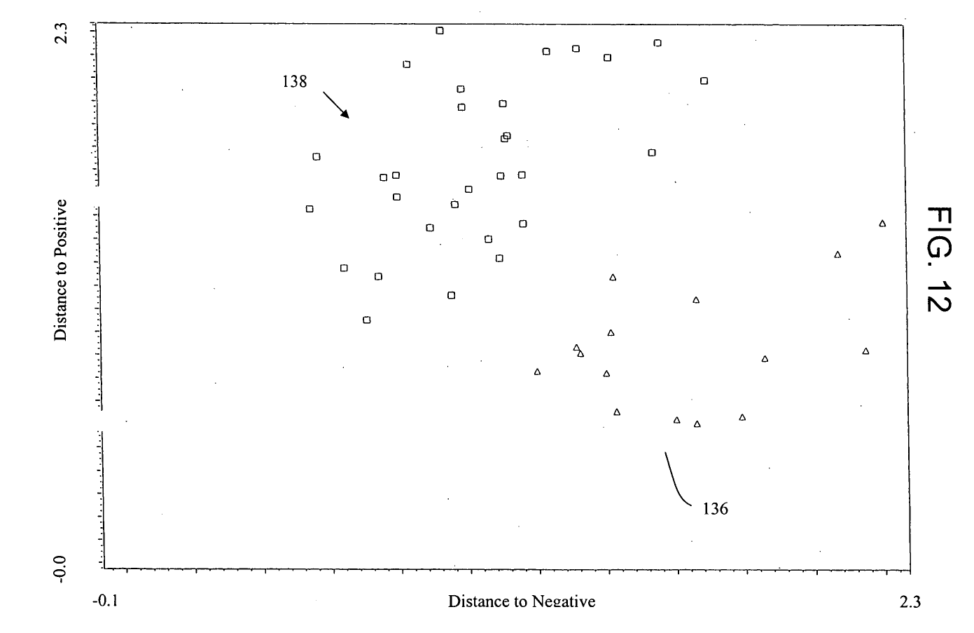

The Mahalanobis distance for each validation sample in the validation set was determined from the PC scores of validation sample and the mean center PC scores of the scrapie-positive cluster grouping of the calibration data set. Likewise, the Mahalanobis distance for each validation sample in the validation set was determined from the PC scores of validation sample and the mean center PC scores of the scrapie-negative cluster grouping of the calibration data set. If the Mahalanobis distance from the validation sample's PC scores to the mean center scrapie-positive PC scores was closer than the Mahalanobis distance from the validation sample's PC scores to the mean center scrapie-negative PC scores, the validation sample was classified as positive for scrapie. Likewise, if the Mahalanobis distance from the validation sample's PC scores to the mean center scrapie-negative PC scores was closer than the Mahalanobis distance from the validation sample's PC scores to the mean center scrapie-positive PC scores, the validation sample was classified as scrapie-negative. FIG. 12 illustrates a scatter plot of the Mahalanobis distances for each validation sample, along with the calibration data points. As can be seen, the validation samples fall into the two distinct clusters of groups, corresponding to a scrapie-positive disease state (triangular data points 136) and scrapie-negative disease state (square data points 138).

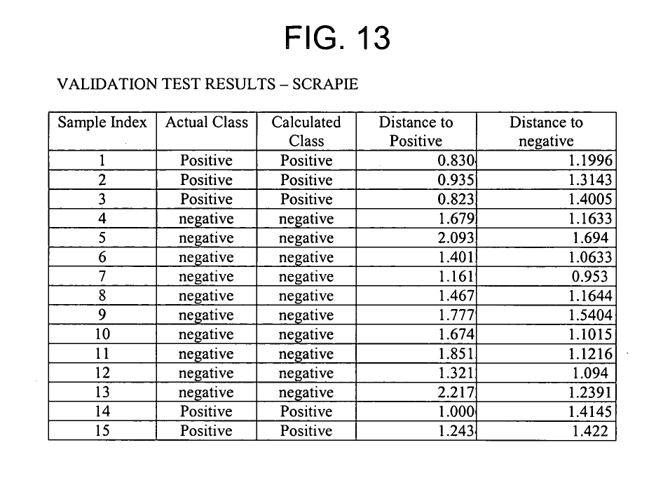

As illustrated in FIG. 13, each validation sample in the set of 15 samples were correctly diagnosed, thus, use of the present methodology revealed 100% agreement of the validation test results with the IHC results for each sample in the validation set.

### Example 4

### Development of a Calibration Model for Diagnosing Scrapie in Whole Blood

Sample Sources

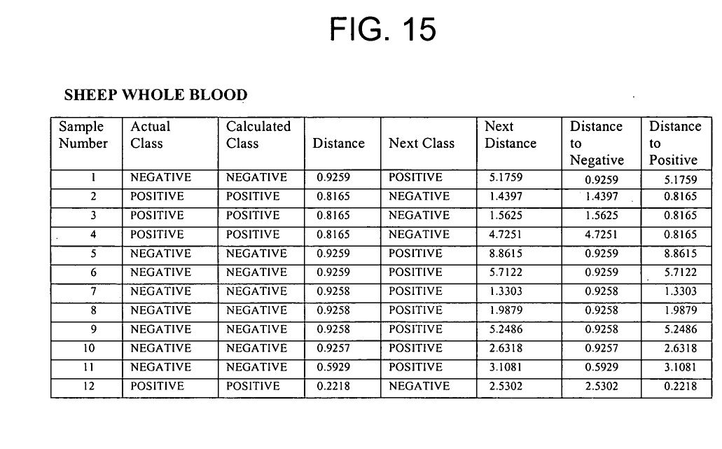

With respect to the calibration model data, 3 known whole blood samples positive for naturally occurring scrapie and 7 known negative whole blood samples were provided by the U.S. Department of Agriculture/Agricultural Research Service in Pullman, Wash. With respect to the validation data, 1 known positive, whole blood sample and 1 known negative, whole blood sample (sample numbers 11 and 12 on FIG. 15) were obtained. In all samples, a scrapie diagnosis was predetermined using standard gold standard methods such as by Western Blot assay or IHC.

It will be appreciated by those skilled in the art that although previously frozen samples are used in the present example, freshly drawn blood samples from live animals or freshly drawn samples from animals prior to slaughtering/sacrificing can be used in the present invention.

Testing

Approximately about 10 μl of untreated, whole blood from each sample subject was utilized. Blood samples were pipetted directly onto the optical platform of the instrument. Each sample in both the calibration set and the validation set were scanned 64 times and co-added to increase the signal to noise ratio, thereby enhancing the spectral absorbance features of the sample. Complete scanning of each sample (including the 64 scans of the same sample) was performed in less than about one minute. The FTIR data collection software, OMNIC/TQ Analyst (© 2002 Thermo Electron Corporation), collected the dispersive spectra for each sample.

FTIR Calibration for Scrapie in Sheep

Second derivatives were calculated from the original spectra for each sample in the calibration data set using a Savitzky-Golay smoothing algorithm using the aforementioned software. Calibration of the test for scrapie was then performed using discriminate principal component analysis performed with the analysis software described herein. As in Examples 1 through 3, it will be appreciated by those skilled in the art that other multivariate chemometric techniques may be used for data analysis in developing the calibration/reference data set.

The software was calibrated with the known scrapie-positive and scrapie-negative samples to develop a calibration model specific for scrapie infections in whole blood taken from sheep. Principal component analysis was performed on the calibration spectral data over the range from about 4500 cm⁻¹ to about 350 cm⁻¹ for each blood sample in the calibration data set. As illustrated in Table 4, nine (9) principal components, accounting for about 99% of the variance in the calibration spectral data set were used for the calibration model.

TABLE 4
PRINCIPAL COMPONENT ANALYSIS
OF CALIBRATION DATA SET

| PRINCIPAL | | CUMULATIVE |
|---|---|---|
| COMPONENT | % VARIANCE | VARIANCE |
| 1 | 63.605 | 63.605 |
| 2 | 17.623 | 81.228 |
| 3 | 6.354 | 87.582 |
| 4 | 3.825 | 91.408 |
| 5 | 2.653 | 94.061 |
| 6 | 1.667 | 95.728 |
| 7 | 1.512 | 97.240 |
| 8 | 0.979 | 98.220 |
| 9 | 0.769 | 98.989 |

A set of PC scores for each tissue sample in the calibration spectral data set was calculated. In addition, the mean center PC scores were generated for the samples known to be positive for scrapie and the samples known to be negative.

The calibration set was cross-validated by calculating the Mahalanobis distances from each known sample in the calibration set to the mean center scrapie-positive PC scores and the mean center scrapie-negative PC scores of the remaining known samples in the calibration set (e.g. by rotating each cross-validated sample out of the calibration set and calculating the Mahalanobis distances for that sample from the mean center scrapie-positive PC scores and the mean center scrapie-negative PC scores of the remaining known samples in the calibration set.)

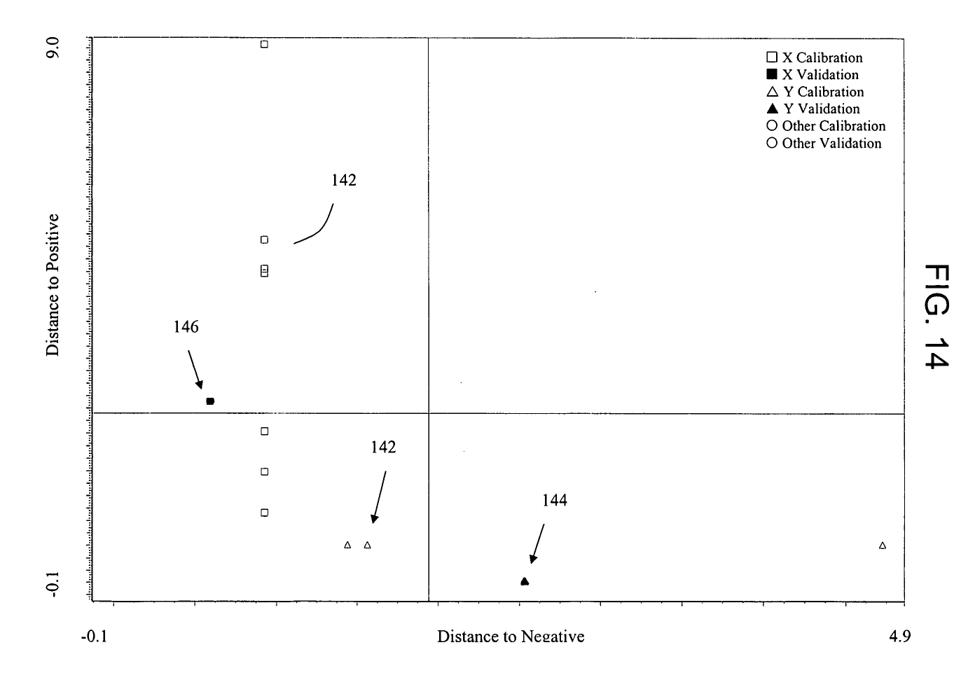

As illustrated in FIG. 14, a scatter plot of the Mahalanobis distances for each calibration sample spectra depicts two distinct clusters of calibration data points (unshaded points), a first cluster grouping 140 corresponding to scrapie-positive tissue samples (triangular unshaded points) and a second cluster grouping corresponding 142 to scrapie-negative tissue samples (square unshaded points). FIG. 15 contains a tabular representation of the calibration data samples (sample numbers 1 through 10) illustrating the actual class and the calculated class for each sample of the calibration data set. All calibration samples were correctly cross-validated as either scrapie-positive or scrapie-negative.

Validation of the Calibration Set for the Method for Detecting Scrapie in Whole Blood

Analysis of Validation Spectral Data Using the Predictive Model

Second derivatives were calculated from the original spectra for each sample in the validation data set using a Savitzky-Golay smoothing algorithm using the aforementioned software. Principal component analysis was then performed on the spectral data for each validation sample using the ten principal components identified in the calibration model. These spectral features were analyzed by PCA and the PC scores for each validation sample were determined.

The Mahalanobis distance for each validation sample in the validation set was determined from the PC scores of validation sample and the mean center PC scores of the scrapie-positive cluster grouping of the calibration data set. Likewise, the Mahalanobis distance for each validation sample in the validation set was determined from the PC scores of validation sample and the mean center PC scores of the scrapie-negative cluster grouping of the calibration data set. If the Mahalanobis distance from the validation sample's PC scores to the mean center scrapie-positive PC scores was closer than the Mahalanobis distance from the validation sample's PC scores to the mean center scrapie-negative PC scores, the validation sample was classified as positive for scrapie. Likewise, if the Mahalanobis distance from the validation sample's PC scores to the mean center scrapie-negative PC scores was closer than the Mahalanobis distance from the validation sample's PC scores to the mean center scrapie-positive PC scores, the validation sample was classified as scrapie-negative. FIG. 14 illustrates a scatter plot of the Mahalanobis distances for each validation sample (shaded points), along with the calibration data points (unshaded points). As can be seen, each validation sample falls into one of the two distinct clusters of groups, corresponding to either a scrapie-positive (sample number 12, triangular shaded point 144) or scrapie-negative (sample number 11, square shaded point 146) disease state.

As illustrated in FIG. 15, each validation sample was correctly diagnosed, thus, use of the present methodology revealed 100% agreement of the validation test results with the IHC results for each sample in the validation set.

### Example 5A

### Development of a Calibration Model for Diagnosing Scrapie in Blood Serum

Sample Sources

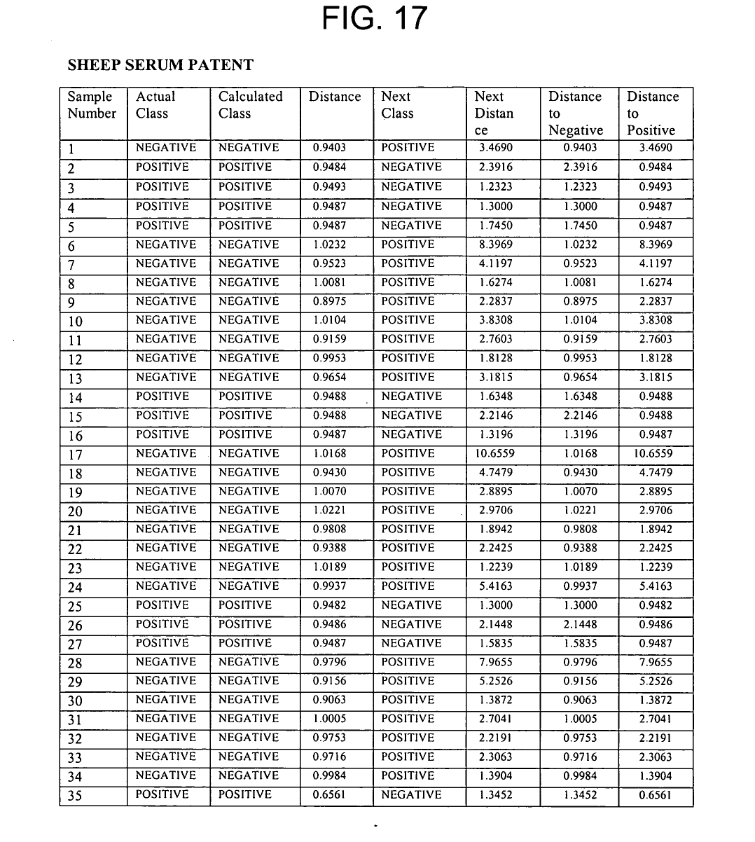

With respect to the calibration model data, 23 known blood serum samples positive for naturally occurring scrapie and 10 known negative blood serum samples were obtained from the U.S. Department of Agriculture/Agricultural Research Service in Pullman, Wash. With respect to the validation data, 1 known positive, blood serum sample and 1 known negative, blood serum sample (sample numbers 9 and 35 on FIG. 17) were also obtained.

In all samples, a scrapie diagnosis was predetermined using standard gold standard methods such as by Western Blot assay or IHC.

Testing

Approximately about 10 μl of untreated, blood serum from each sample subject was utilized. Blood samples were pipetted directly onto the optical platform of the instrument. Each sample in both the calibration set and the validation set were scanned as described with reference to Example 4.

FTIR Calibration for Scrapie in Sheep

Second derivatives were calculated from the original spectra for each sample in the calibration data set using a Savitzky-Golay smoothing algorithm using the aforementioned software. Calibration of the test for scrapie was then performed using discriminate principal component analysis performed with the analysis software described herein.

The software was calibrated with the known scrapie-positive and scrapie-negative samples to develop a calibration model specific for scrapie infections in blood serum taken from sheep. Principal component analysis was performed on the calibration spectral data over the range from about 4500 cm⁻¹ to about 350 cm⁻¹ for each serum sample in the calibration data set. As illustrated in Table 5, fifteen (15) principal components, accounting for over 99% of the variance in the calibration spectral data set were used for the calibration model.

TABLE 5
PRINCIPAL COMPONENT ANALYSIS
OF CALIBRATION DATA SET

| PRINCIPAL | | CUMULATIVE |
|---|---|---|
| COMPONENT | % VARIANCE | VARIANCE |
| 1 | 90.192 | 90.192 |
| 2 | 4.207 | 94.400 |
| 3 | 2.420 | 96.819 |
| 4 | 0.663 | 97.482 |
| 5 | 0.439 | 97.921 |
| 6 | 0.324 | 98.245 |
| 7 | 0.254 | 98.499 |
| 8 | 0.184 | 98.683 |
| 9 | 0.180 | 98.863 |
| 10 | 0.152 | 99.016 |
| 11 | 0.126 | 99.142 |
| 12 | 0.103 | 99.245 |
| 13 | 0.092 | 99.337 |
| 14 | 0.088 | 99.426 |
| 15 | 0.073 | 99.499 |

A set of PC scores for each tissue sample in the calibration spectral data set was calculated. In addition, the mean center PC scores were generated for the samples known to be positive for scrapie and the samples known to be negative.

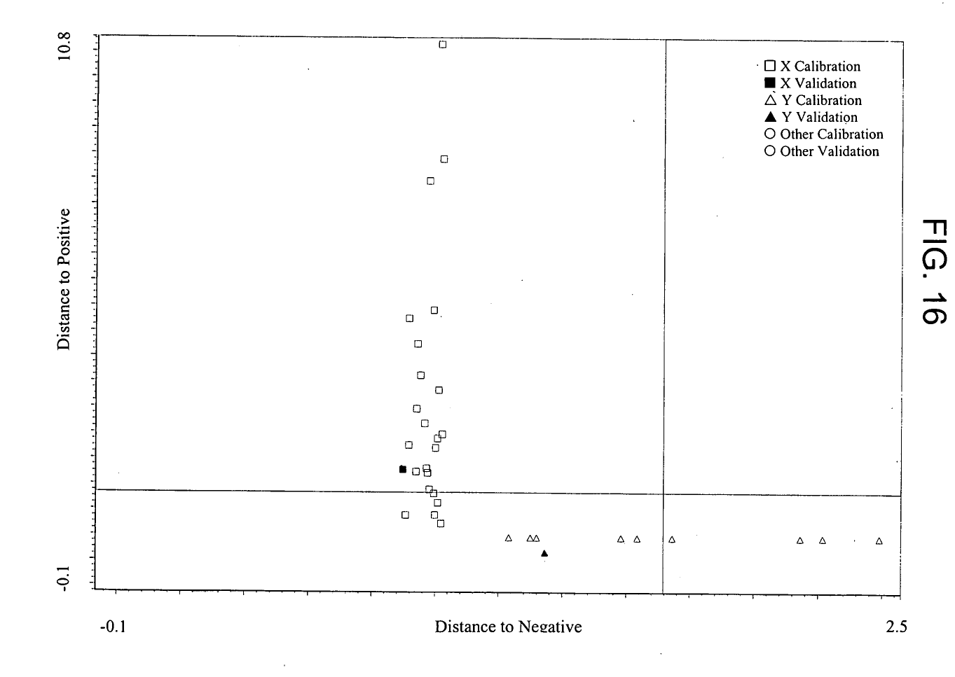

The calibration set was cross-validated as described with reference to Example 4. As illustrated in FIG. 16, a scatter plot of the Mahalanobis distances for each calibration sample spectra depicts two distinct clusters of calibration data points (unshaded points), a first cluster grouping 150 corresponding to scrapie-positive tissue samples (triangular unshaded points) and a second cluster grouping 152 corresponding to scrapie-negative tissue samples (square unshaded points). FIG. 17 contains a tabular representation of the calibration data samples (sample numbers 1 through 8 and 10 through 34) illustrating the actual class and the calculated class for each sample of the calibration data set. All calibration samples were correctly cross-validated as either scrapie-positive or scrapie-negative.

Validation of the Calibration Set for the Method for Detecting Scrapie in Blood Serum

Analysis of Validation Spectral Data Using the Predictive Model

Second derivatives were calculated from the original spectra for each sample in the validation data set using a Savitzky-Golay smoothing algorithm using the aforementioned software. Principal component analysis was then performed on the spectral data for each validation sample using the ten principal components identified in the calibration model. These spectral features were analyzed by PCA and the PC scores for each validation sample were determined.

The Mahalanobis distance for each validation sample in the validation set was determined as described with reference to Example 4. FIG. 16 illustrates a scatter plot of the Mahalanobis distances for each validation sample (shaded points), along with the calibration data points (unshaded points). As can be seen, each validation sample falls into one of the two distinct clusters of groups, corresponding to either a scrapie-positive (sample number 35, triangular shaded point 154) or scrapie-negative (sample number 9, square shaded point 156) disease state.

As illustrated in FIG. 17, each validation sample was correctly diagnosed, thus, use of the present methodology revealed 100% agreement of the validation test results with the IHC results for each sample in the validation set.

### Example 5B

### Sample Scoring

Each calibration and validation sample in Example 5A was scored for the presence of scrapie in the sample. As illustrated in FIG. 18, a score was calculated for each sample by subtracting the distance to positive from the distance to negative. If a sample score falls within the range of −0.1000 to 0.0000, the sample is classified as <suspect positive>. As shown in FIG. 18, none of the samples tested were classified as <suspect positive> samples.

### Example 6

### Development of a Calibration Model for Diagnosing Scrapie in Blood Plasma

Sample Sources

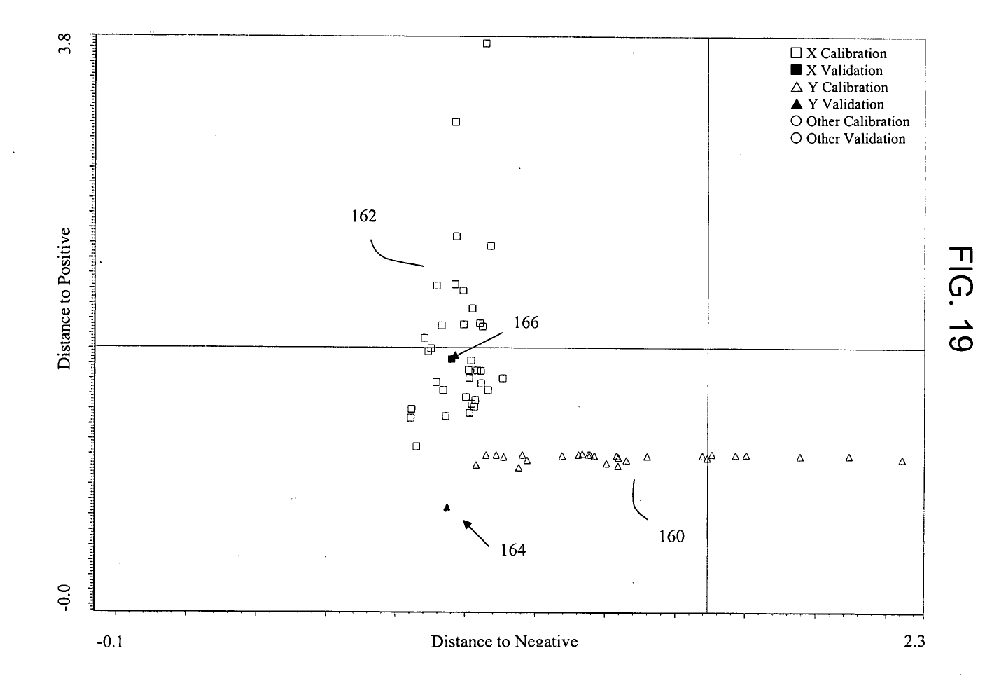

With respect to the calibration model data, 27 known blood plasma samples positive for naturally occurring scrapie and 34 known negative blood plasma samples were obtained from the U.S. Department of Agriculture/Agricultural Research Service in Pullman, Wash. With respect to the validation data, 1 known positive, blood plasma sample and 1 known negative, blood plasma sample (sample numbers 33 and 53 on FIG. 19) were also provided.

In all samples, a scrapie diagnosis was predetermined using standard gold standard methods such as by Western Blot assay or IHC.

Testing

Approximately about 10 μl of untreated, blood plasma from each sample subject was utilized. Blood plasma samples were pipetted directly onto the optical platform of the instrument. Each sample in both the calibration set and the validation set were scanned as described with reference to Example 4.

FTIR Calibration for Scrapie in Sheep

Second derivatives were calculated from the original spectra for each sample in the calibration data set using a Savitzky-Golay smoothing algorithm using the aforementioned software. Calibration of the test for scrapie was then performed using discriminate principal component analysis performed with the analysis software described herein.

The software was calibrated with the known scrapie-positive and scrapie-negative samples to develop a calibration model specific for scrapie infections in blood plasma taken from sheep. Principal component analysis was performed on the calibration spectral data over the range from about 4500 cm⁻¹ to about 350 cm⁻¹ for each plasma sample in the calibration data set. As illustrated in Table 6, twenty-four (24) principal components, accounting for over 96% of the variance in the calibration spectral data set were used for the calibration model.

TABLE 6
PRINCIPAL COMPONENT ANALYSIS
OF CALIBRATION DATA SET

| PRINCIPAL | | CUMULATIVE |
|---|---|---|
| COMPONENT | % VARIANCE | VARIANCE |
| 1 | 31.476 | 31.476 |
| 2 | 20.793 | 52.270 |
| 3 | 14.990 | 67.260 |
| 4 | 5.604 | 72.864 |
| 5 | 3.489 | 76.353 |
| 6 | 3.282 | 79.635 |
| 7 | 2.900 | 82.535 |
| 8 | 2.062 | 84.596 |
| 9 | 1.822 | 86.419 |
| 10 | 1.546 | 87.965 |
| 11 | 1.169 | 89.134 |
| 12 | 1.148 | 90.283 |
| 13 | 0.994 | 91.277 |
| 14 | 0.720 | 91.997 |
| 15 | 0.745 | 92.742 |
| 16 | 0.638 | 93.380 |
| 17 | 0.620 | 93.999 |
| 18 | 0.541 | 94.540 |
| 19 | 0.468 | 95.008 |
| 20 | 0.377 | 95.385 |
| 21 | 0.371 | 95.756 |
| 22 | 0.315 | 96.071 |
| 23 | 0.284 | 96.355 |
| 24 | 0.281 | 96.637 |

A set of PC scores for each tissue sample in the calibration spectral data set was calculated. In addition, the mean center PC scores were generated for the samples known to be positive for scrapie and the samples known to be negative.

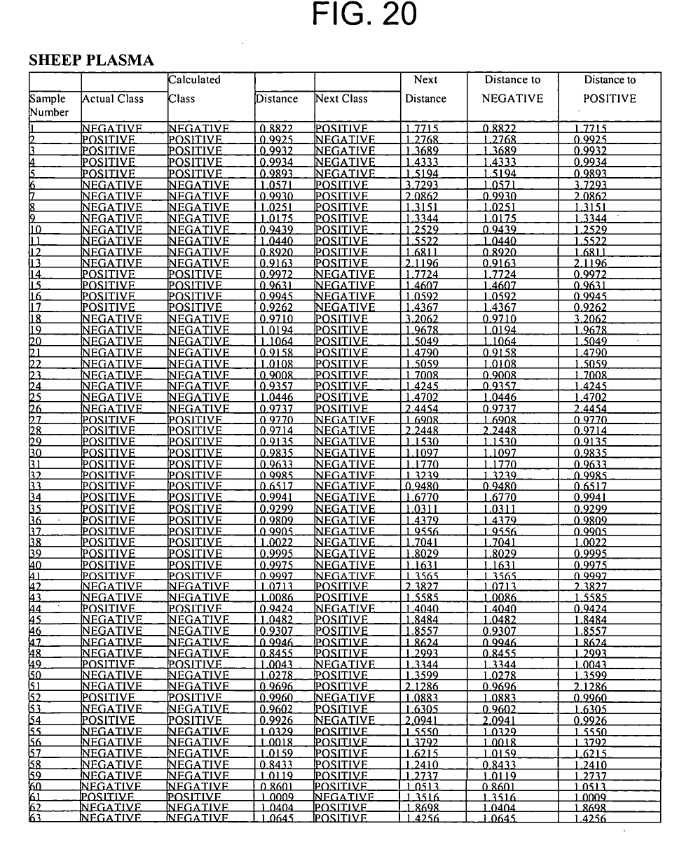

The calibration set was cross-validated as described with reference to Example 4. As illustrated in FIG. 19, a scatter plot of the Mahalanobis distances for each calibration sample spectra depicts two distinct clusters of calibration data points (unshaded points), a first cluster grouping 160 corresponding to scrapie-positive samples (triangular unshaded points) and a second cluster grouping 162 corresponding to scrapie-negative tissue samples (square unshaded points). FIG. 20 contains a tabular representation of the calibration data samples (sample numbers 1 through 32, 34 through 52, and 54 through 63) illustrating the actual class and the calculated class for each sample of the calibration data set. All calibration samples were correctly cross-validated as either scrapie-positive or scrapie-negative.

Validation of the Calibration Set for the Method for Detecting Scrapie in Blood Plasma

Analysis of Validation Spectral Data Using the Predictive Model

Second derivatives were calculated from the original spectra for each sample in the validation data set using a Savitzky-Golay smoothing algorithm using the aforementioned software. Principal component analysis was then performed on the spectral data for each validation sample using the ten principal components identified in the calibration model. These spectral features were analyzed by PCA and the PC scores for each validation sample were determined.

The Mahalanobis distance for each validation sample in the validation set was determined as described with reference to Example 4. FIG. 19 illustrates a scatter plot of the Mahalanobis distances for each validation sample (shaded points), along with the calibration data points (unshaded points). As can be seen, each validation sample falls into one of the two distinct clusters of groups, corresponding to either a scrapie-positive (sample number 33, triangular ushaded point 164) or scrapie-negative (sample number 53, square shaded point 164) disease state.

As illustrated in FIG. 20, each validation sample was correctly diagnosed, thus, use of the present methodology revealed 100% agreement of the validation test results with the IHC results for each sample in the validation set.

1. A method for developing a calibration model for use in differentially diagnosing naturally occurring chronic wasting disease (CWD) in a cervid, said method comprising:
obtaining a plurality of known CWD-positive and known CWD-negative tissue samples to form a calibration set;
analyzing each of the known CWD-positive tissue samples and each of the known CWD-negative tissue samples in the calibration set using an IR spectrometric method;
obtaining at least one set of calibration IR spectral data for each of the known CWD-positive and known CWD-negative tissue samples in the calibration set; and
applying a multivariate chemometric technique to the calibration IR spectral data for each of the known CWD-positive tissue samples and each of the known CWD-negative tissue samples to statistically differentiate the CWD-positive and CWD-negative calibration IR spectral data contained in the calibration set.

2. The method of claim 1, wherein the tissue sample substantially untreated.

3. The method of claim 1, wherein each of the known CWD-positive and the known CWD-negative tissue samples are lymph tissue samples including a substantial portion of primary follicles, secondary follicles or a combination thereof.

4. The method of claim 1, wherein the spectrometric method is one of an absorption and a reflectance spectrometric method.

5. The method of claim 1, wherein each of the known CWD-positive and known CWD-negative tissue samples in the calibration set is analyzed at wavenumbers from about 4500 cm⁻¹ to about 350 cm⁻¹.

6. The method of claim 1, wherein principal component analysis is applied to the calibration spectral data obtained for each of the known CWD-positive tissue samples and each of the known CWD-negative tissue samples to statistically differentiate the CWD-positive and CWD-negative calibration spectral data contained in the calibration set.

7. The method of claim 6, further comprising calculating CWD-positive mean center principal component scores for the known CWD-positive tissue samples and CWD-negative mean center principal component scores for the known CWD-negative tissue samples.

8. The method of claim 7, further comprising analyzing an unknown tissue sample using a spectrometric method, obtaining at least one set of spectral data for the unknown tissue sample, calculating the principal component scores of the unknown tissue sample, comparing the principal component scores of the unknown sample to the CWD-positive mean center principal component scores and the CWD-negative mean center principal component scores; and classifying the unknown tissue sample as one of CWD-positive and CWD-negative.

9. The method of claim 8, further comprising calculating a Mahalanobis distance from the principal component scores of the unknown sample to each of the CWD-positive and CVD-negative mean centered principal component scores, wherein the unknown tissue sample is classified as CWD-positive, CWD-negative or a Mahalanobis outlier.

10. The method of claim 1, further comprising normalizing each of said sets of spectral data prior to applying said multivariate chemometric technique to the calibration spectral data to reduce noise and adjust for drift and diffuse light scatter.

11. The method of claim 1, further comprising analyzing an unknown, untreated tissue sample using a spectrometric method, obtaining at least one set of spectral data for the unknown tissue sample, applying a multivariate chemometric technique to the spectral data for the unknown tissue sample, comparing the spectral data of the unknown sample to the calibration spectral data of the known CWD-positive and the known CWD-negative tissue samples, whereby said unknown sample is classified as one of CWD-positive and CWD-negative.

12. The method of claim 11, further comprising updating the calibration set with the spectral data obtained for said unknown tissue sample.

13. A method for detecting a transmissible spongiform encephalopathy (TSE) in a cervid, sheep or goat, said method comprising:
selecting a raw, unknown sample from a live or postmortem subject;
obtaining at least one set of IR spectral data from the sample; and
comparing the IR spectral data of the sample with a calibration model comprising IR spectral data from a plurality of known TSE-positive and known TSE-negative samples of similar type from the same species as the subject to determine whether the sample is TSE-positive or TSE-negative.

14. The method of claim 13, wherein the untreated, unknown sample is selected from the group consisting of lymph tissue, brain tissue and blood.

15. The method of claim 13, wherein infrared absorbance spectra for the unknown sample and each of the known samples are obtained at wavenumbers from about 7400 cm⁻¹ to about 350 cm⁻¹.

16. The method of claim 13, wherein the calibration model includes spectral data from a plurality of known TSE-positive and known TSE-negative samples subjected to a spectral data compression technique selected from the group consisting of Principal Component Analysis (PCA), Partial Least Squares Regression (PLS), Principal Component Regression (PCR), Multiple Linear Regression (MLR) and Discriminant Analysis.

17. The method of claim 16, wherein the spectral data of the unknown sample is subjected to a spectral data compression technique and wherein the compressed spectral data of the unknown sample is compared to the calibration model, wherein the unknown sample is classified as TSE-positive or TSE-negative.

18. A method of detecting a change in spectral response between diseased and normal samples to differentially diagnose a transmissible spongiform encephalopathy (TSE) in a cervid, sheep or goat, said method comprising:
obtaining a sample from at least one of a lymph node containing cortex or paracortex tissue, brain tissue or blood from a subject;
directing a beam of IR light at wavenumbers from about 7400 cm⁻¹ to about 350 cm⁻¹ to produce IR spectral data for the sample;
comparing the IR spectral data for the sample with a calibration set of IR spectral data comprising both a TSE-positive and a TSE-negative predictive model; and
determining whether variation in absorption occurs in the sample, the variation being characteristic of one of a TSE-positive or a TSE-negative condition.

19. The method of claim 18, wherein the calibration set of spectral data is obtained through multivariate analysis of known TSE-positive and known TSE-negative samples from the same species as the subject.

20. The method of claim 19, wherein the spectral data of the unknown sample is subjected to multivariate analysis and compared to the calibration set of spectral data, whereby the variation in absorption in the unknown sample indicates one of a TSE-positive or a TSE-negative condition.

21. A method for rapidly screening unknown samples for a TSE in cervids, sheep or goats, said method comprising:
analyzing an untreated sample selected from lymph tissue, brain tissue or plasma using an IR spectroscopic method providing IR spectral data;
applying principal component analysis to the IR spectral data obtained from the unknown sample and calculating the principal component scores for the unknown sample; and
differentially determining whether the sample is TSE-positive or TSE-negative by comparing the principal component scores for the unknown sample with a calibration IR spectral data set comprising a set of mean centered principal component scores for each of a known TSE-positive sample grouping and a known TSE-negative sample grouping from the same species as the subject.

22. The method of claim 21, wherein spectral data for the unknown sample and each of the known samples are obtained at wavenumbers from about 7400 cm⁻¹ to about 350 cm⁻¹.

23. The method of claim 21, wherein differentially determining whether the unknown sample is TSE-positive or TSE-negative includes calculation of a Mahalanobis distance from the principal component scores of the unknown sample to the mean centered principal component scores for each of the TSE-positive and the TSE-negative sample groupings in the calibration spectral data set and determining whether the Mahalanobis distance from the principal component scores of the unknown sample is closer to the mean center TSE-positive principal component scores or closer to the mean center TSE-negative principal component scores.

24. The method of claim 21, further comprising confirming of a TSE-positive diagnosis using a secondary diagnosis method selected from the group consisting of immunohistochemistry assay, Western Blot assay and microscopic examination.

25. A method for monitoring Chronic Wasting Disease within a particular region, state or country, the method comprising:
presenting a hunter harvested or naturally postmortem animal species;
recording the originating location of the postmortem animal;
obtaining a raw, untreated sample from one of a lymph node, brain tissue or blood plasma of the animal;
labeling the sample with computer readable indicia including at least one of the location of the animal, hunter information and the date;
obtaining IR spectral data for the sample at wavenumbers from about 7400 cm⁻¹ to about 350 cm⁻¹,
comparing the IR spectral data to a reference set of IR spectral data; and
differentially identifying the sample as either CWD-positive or CWD-negative as indicated by the reference set of IR spectral data.

26. The method of claim 25, wherein the spectral data of the sample is obtained using Fourier transform infrared spectroscopy.

27. The method of claim 25, wherein the reference set of spectral data is developed by: analyzing a plurality of known CWD-positive samples and a plurality of known CWD-negative samples using a spectrometric method at wavenumbers from about 7400 cm⁻¹ to about 350 cm⁻¹ and generating predicted scores for each of the CWD-positive and CWD-negative sample groupings using a multivariate technique.

28. The method of claim 27, wherein predicted scores for the unknown sample are compared with the CWD-positive predicted scores and CWD-negative predicted scores to identify the sample as either CWD-positive or CWD-negative.

29. The method of claim 25, further comprising incorporating IR spectral data from a correctly diagnosed sample into the reference set of spectral data.
